(19) United States
(12) Patent Application Publication
(10) Pub. No.: US 2005/0171101 A1
(43) Pub. Date: Aug. 4, 2005

(54) Phenanthridinones as parp inhibitors

(75) Inventors: Hirofumi Yamamoto, Osaka (JP); Koichiro Mukoyoshi, Osaka (JP); Kouji Hattori, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka (JP)

(21) Appl. No.: 10/508,004

(22) PCT Filed: Mar. 25, 2003
(86) PCT No.: PCT/JP03/03579

(30) Foreign Application Priority Data
Mar. 26, 2002 (AU) PS 1374

(51) Int. Cl. A61K 31/5377; A61K 31/496; A61K 31/473; C07D 413/02; C07D 43/02

(52) U.S. Cl. 514/232.5; 514/253.03; 514/290; 544/125; 544/361; 546/79

(57) ABSTRACT

A compound of the formula (I): wherein ring A is a carbocyclic group, R1 is hydrogen or a halogen atom or a lower alkyl group, R2 is a di(lower)alkylamino group or N-containing heterocyclic group, among which the N-containing heterocyclic group may be substituted with one or more substituent(s), Y is an oxygen or sulfur atom, n is an integer from 0 to 2, and m is an integer from 0 to 4, or its prodrug, or their salt which has poly(adenosine 5′-diphospho-ribose)polymerase inhibiting activity.

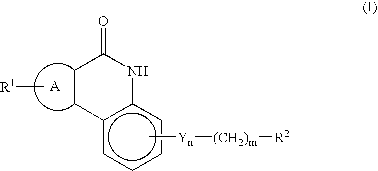

### TECHNICAL FIELD

This invention relates to novel tricyclic compounds having a pharmacological activity, a process for their production and a pharmaceutical composition containing the same.

### BACKGROUND ART

Poly(adenosine 5′-diphospho-ribose)polymerase (hereinafter called as PARP) is an enzyme located in the nuclei of cells of various organs, including muscle, heart and brain cells. After recognizing strand breaks of DNA caused by NMDA(N-methyl-D-aspartate), NO, active oxygen and the like, PARP catalyzes the attachment reaction of ADP-ribose units of nicotinamide adenine dinucleotide (NAD) to a variety of nuclear proteins, including histones and PARP itself. However, excess activation of PARP leads to depletion of NAD and ATP in cells to induce cell death. Therefore, the PARP inhibitors are expected to be useful in treatment and prevention of various diseases ascribed by NMDA- and NO-induced toxicity.

Some benimidazole derivatives having inhibitory activity of PARP have been known, for example, in WO00/29384, WO00/32579, WO00/68206 and WO01/21615.

### DISCLOSURE OF INVENTION

An object of this invention is to provide novel tricyclic compounds, particularly phenanthridiones and tetrahydrophenanthridinones, and salts thereof.

Another object of this invention is to provide a process for the production of the tricyclic compounds and salts thereof.

A further object of this invention is to provide a pharmaceutical composition containing an effective amount of the tricyclic compound, its prodrug or a pharmaceutically acceptable salt thereof, which has a PARP inhibiting activity, as an active ingredient in admixture of a pharmaceutically acceptable carrier.

Still further object of this invention is to provide a use of the tricyclic compound, its prodrug or a pharmaceutical acceptable salt thereof for preparing a medicament for treating or preventing diseases ascribed by excess activation of PARP.

Still further object of the invention is to provide a method of treating or preventing diseases ascribed by excess activation of PARP by administering the tricyclic compound, its prodrug or a pharmaceutical acceptable salt thereof in an effective amount to inhibit PARP activity.

The tricyclic compounds of this invention are represented by the following formula (I): 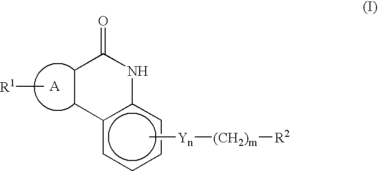
wherein
ring A is a carbocyclic group,
R¹ is hydrogen or a halogen atom or a lower alkyl group,
R² is a di(lower)alkylamino group or N-containing heterocyclic group, among which the N-containing heterocyclic group may be substituted with one or more substituent(s),
Y is an oxygen or sulfur atom,
n is an integer from 0 to 2, and
m is an integer from 0 to 4.

Suitable examples and illustrations of the above definitions are explained in detail as follows.

The term “lower” means a group having 1 to 6 carbon atom(s), unless otherwise provided.

The term “one or more” means 1 to 6, preferably 1 to 3, and more preferably 1 or 2.

Suitable examples of the lower alkyl group and the lower alkyl moiety in the di(lower)alkylamino group are straight or branched ones having 1 to 6 carbon atoms such as methyl, ethyl, n-propyl, isopropyl, n-butyl, 2-ethylbutyl, isobutyl, tert-butyl, pentyl, n-hexyl, etc.

Suitable examples of the halogen atom are fluorine, chlorine, bromine or iodine.

Suitable examples of the carbocyclic group are cyclo(lower)alkane ring (e.g., cyclobutane, cyclopentane, cyclohexane or cycloheptane), cyclo(lower)alkene ring (e.g., cyclopentene or cyclohexene) and aromatic hydrocarbon ring (e.g., benzene or naphthalene).

Suitable examples of the N-containing heterocyclic group are monocyclic or condensed heterocyclic groups containing 1 to 4 nitrogen atom(s) and optionally 1 to 2 oxygen or sulfur atom.

Preferable examples of the N-containing heterocyclic group are:
(1) unsaturated 3 to 7-membered, preferably 5- or 6-membered heteromonocyclic group containing 1 to 4 nitrogen atoms, for example, pyrrolyl, pyrrolinyl, imidazolyl, pyrazolyl, pyridyl, tetrahydropyridyl, pyrimidinyl, tetrahydropyrimidinyl, pyrazinyl, pyridazinyl, triazolyl (e.g., 4H-1,2,4-triazolyl, 1H-1,2,3-triazolyl or 2H-1,2,3-triazolyl) or tetrazolyl (e.g., 1H-tetrazolyl or 2H-tetrazolyl),
(2) saturated 3 to 7-membered, preferably 5- or 6-membered heteromonocyclic group containing 1 to 4 nitrogen atoms, for example, pyrrolidinyl, imidazolidinyl, piperidyl or piperazinyl,
(3) unsaturated 3 to 7-membered, preferably 5- or 6-membered heteromonocyclic group containing 1 to 3 nitrogen atoms and 1 to 2 oxygen atoms, for example, oxazolyl, isoxazolyl or oxadiazolyl (e.g., 1,2,4-oxadiazolyl, 1,2,4-oxadiazolinyl, 1,3,4-oxadiazolyl or 1,2,5-oxadiazolyl);
(4) saturated 3 to 7-membered, preferably 5- or 6-membered heteromonocyclic group containing 1 to 3 nitrogen atoms and 1 to 2 oxygen atoms, for example, morpholinyl,
(5) unsaturated 3 to 7-membered, preferably 5- or 6-membered heteromonocyclic group containing 1 to 3 nitrogen atoms and 1 to 2 sulfur atoms, for example, thiazolyl or thiadiazolyl (e.g., 1,2,4-thiadiazolyl, 1,3,4-thiadiazolyl or 1,2,5-thiadiazolyl),
(6) saturated 3 to 7-membered preferably 5- or 6-membered heteromonocyclic group containing 1 to 3 nitrogen atoms and 1 to 2 sulfur atoms, for example, thiomorpholinyl or thiazolidinyl,
(7) unsaturated condensed heterocyclic group containing 1 to 3 nitrogen atoms, for example, benzopyrrolyl, benzimidazolyl, benzopyrazolyl, benzotriazolyl, quinolyl, isoquinolyl, indolyl, indolinyl, isoindolidinyl, 1,2,3,4-tetrahydroquinolyl or pyrido[3,4-b]indolyl,
(8) unsaturated condensed heterocyclic group containing 1 to 3 nitrogen atoms and 1 to 2 oxygen atoms, for example, benzoxazolyl, benzoxadiazolyl or phenoxazinyl; or
(9) unsaturated condensed heterocyclic group containing 1 to 3 nitrogen atoms and 1 to 2 sulfur atoms, for example, benzothiazolyl, benzisothiazolyl or phenothiazinyl.

Among the above, more preferable heterocyclic group is an unsaturated 5- or 6-membered heteromonocyclic group as mentioned in the above (1) or a saturated 5- or 6-membered heteromonocyclic group as mentioned in the above (2) and (4), among which the most preferable one is pyridyl, tetrahydropyridyl, piperidyl, piperazinyl or morpholinyl.

The N-containing heterocyclic group and 1,3,4,9-tetrahydro-2H-β-carbolin-2-yl group may be optionally substituted with one or more substituent(s) such as hydroxy; amino; carboxy; cyano; nitro; carbamoyl; oxo; halogen (e.g., fluorine, bromine or chlorine); lower alkyl (e.g., methyl, ethyl, isopropyl or tert-butyl); lower alkoxy (e.g., methoxy, ethoxy, butoxy or n-propoxy); halo(lower)alkyl (e.g., chloromethyl or trifluoromethyl); optionally substituted aryl [e.g., naphthyl or phenyl which may be further substituted with halogen (e.g., fluorine, bromine or chlorine), lower alkoxy (e.g., methoxy, ethoxy, butoxy or n-propoxy), cyano or halo(lower)alkyl (e.g., chloromethyl or trifluoromethyl)]; aryloxy (e.g., phenoxy); or aroyl (e.g., benzoyl).

Suitable salts of the compound (I) are pharmaceutically acceptable, conventional and non-toxic salts, for example an organic acid addition salt (e.g. formate, acetate, trifluoroacetate, maleate, tartarate, oxalate, methanesulfonate, benzenesulfonate or toluenesulfonate), an inorganic acid addition salt (e.g. hydrochloride, hydrobromide, sulfate or phosphate), a salt with an amino acid (e.g. aspartate or glutamate), or the like.

The compounds (I) may contain one or more asymmetric centers and thus they can exist as enantiomers or diastereoisomers.

The compounds (I) may also exist in tautomeric forms and the invention includes both mixtures and separate individual tautomers.

The compound (I) and its salt can be in a form of a solvate, which is also included within the scope of the present invention. The solvate preferably include a hydrate and an ethanolate.

Also included in the scope of invention are radiolabelled derivatives of compounds (I) which are suitable for biological studies.

The “prodrug” may be a derivative of the compound (I) having a chemically or metabolically degradable group, which becomes pharmaceutically active substance after biotransformation.

Preferred compounds (I) are the ones ring A is a cyclo(lower)alkane ring or aromatic hydrocarbon ring,
R¹ is hydrogen or a halogen atom,
n is an integer of 0 or 1, and
R², Y and m have the same meaning as defined in the above.

More preferred compounds (I) are the ones wherein R² is tetrazolyl, pyridyl, piperidyl, piperazinyl, morpholinyl, isoindolidinyl or pyrido[3,4-b]indolyl, each of which may be substituted with one or more substituent(s).

Further preferred compounds (I) are the ones wherein the ring A is a cyclohexane ring,
R¹ is hydrogen atom, and
R², Y, n and m have the same meaning as defined in the above, and
the ones wherein the ring A is a benzene ring,
R¹ is hydrogen or a halogen atom,
R² and Y have the same meaning as defined in the above,
n is 0, and
m is an integer 3 or 4.

Especially preferred compounds (I) are those wherein the ring A is a cyclohexane ring,
R¹ is hydrogen atom,
R² has the same meaning as defined in the above,
Y is an oxygen atom,
n is an integer of 0 or 1, and
m is an integer from 0 to 3.

The compound (I) or a salt thereof can be prepared by the following processes. 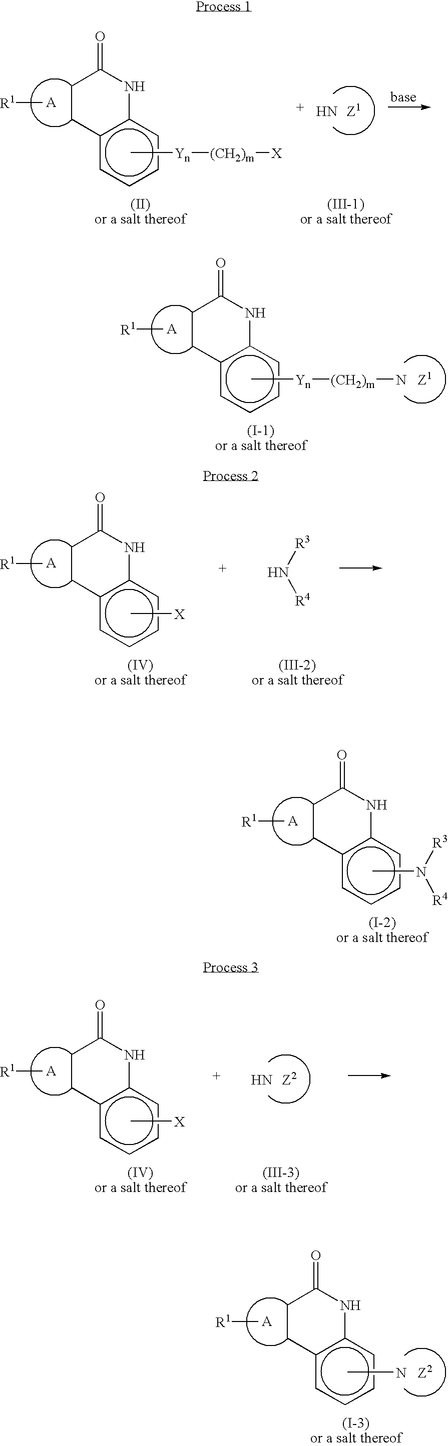 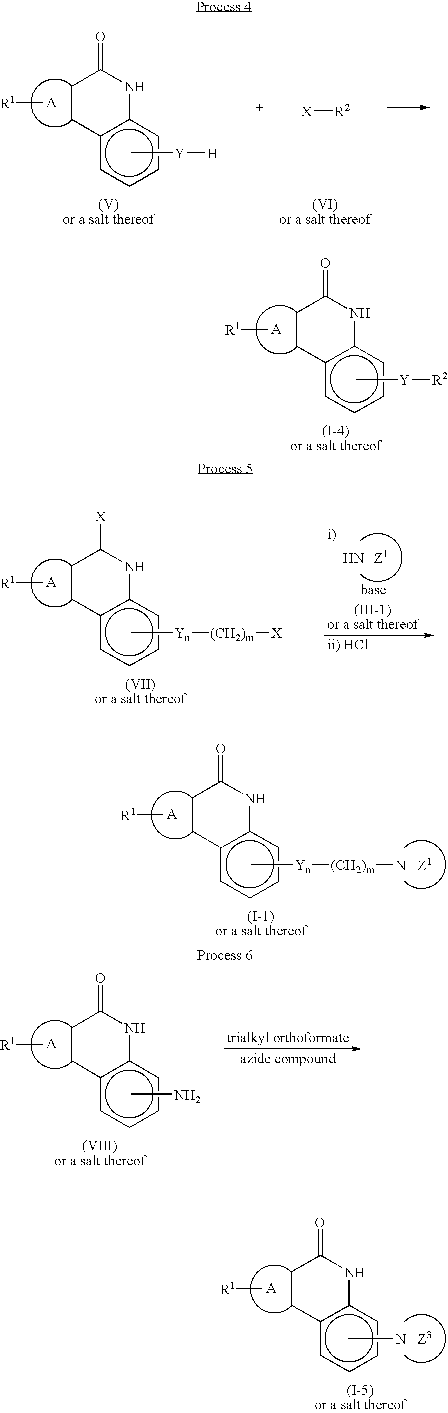 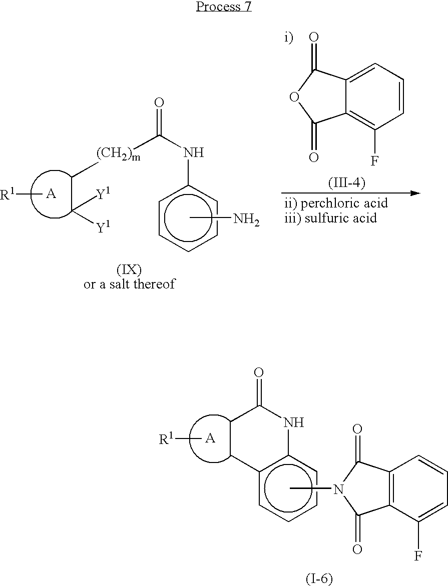
wherein, R¹, R², Y, n, m and the ring A are each as defined above, X is a leaving group, R³ and R⁴ are each lower alkyl group, Y¹s are independently a hydroxy group or oxygen atom and/or together represent an oxo group or ethylene ketal or propylene ketal group, 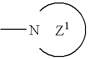
is a N-containing heterocyclic group or 1,3,4,9-tetrahydro-2H-β-carbolin-2-yl group, both of which may be optionally substituted with one or more substituent(s), 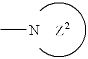
is a N-containing heterocyclic group which may be optionally substituted with one or more substituent(s), 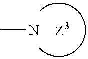
is a tetrazolyl group.

Suitable leaving group may be halogen (e.g., fluoro, chloro, bromo or iodo), arylsulfonyloxy (e.g., benzenesulfonyloxy or tosyloxy), alkylsulfonyloxy (e.g., mesyloxy or ethanesulfonyloxy) or the like, among which the preferable one is halogen.

### PROCESS 1

The object compound (I-1) or its salt can be prepared by reacting a compound (II) or its salt with a compound (III-1) or its salt.

This reaction is usually carried out in the presence of an inorganic or an organic base. Suitable inorganic base may be an alkali metal [e.g., sodium or potassium], an alkali metal hydroxide [e.g., sodium hydroxide or potassium hydroxide], alkali metal hydrogen carbonate [e.g., sodium hydrogen carbonate or potassium hydrogen carbonate], alkali metal carbonate [e.g., sodium carbonate or potassium carbonate], alkaline earth metal carbonate [e.g., calcium carbonate or magnesium carbonate], alkali metal hydride [e.g., sodium hydride or potassium hydride], or the like. Suitable organic base may be tri(lower)alkylamine [e.g., triethylamine or N,N-diisopropylethylamine], alkyl magnesium bromide [e.g., methyl magnesium bromide or ethyl magnesium bromide], alkyl lithium [e.g., methyl lithium or butyl lithium], lithium diisopropylamide, lithium hexamethyldisilazido, or the like.

The reaction is usually carried out in a conventional solvent such as an alcohol [e.g., methanol, ethanol, propanol or isopropanol], aromatic hydrocarbon [e.g., benzene, toluene or xylene], ethyl acetate, acetonitrile, dioxane, chloroform, methylene chloride, N,N-dimethylformamide or any other organic solvent which does not adversely influence the reaction.

The reaction temperature is not critical , and the reaction is usually carried out under cooling to heating.

### PROCESS 2

The object compound (I-2) or its salt can be prepared by reacting a compound (IV) or its salt with a compound (III-2) or its salt.

This reaction is usually carried out in the presence of an inorganic or organic base, a binaphthyl compound and palladium catalyst. Suitable inorganic base may be an alkali metal alkoxide [e.g., sodium methoxide, potassium ethoxide or sodium tert-butoxide], or the like. Suitable binaphthyl compound may be 2,2′-bis(diphenylphophino)-1,1′-binaphthyl. Suitable palladium compound may be tris(dibenzylideneacetone)dipalladium (0).

The reaction is usually carried out in a conventional solvent such as aromatic hydrocarbon [e.g., benzene, toluene or xylenel, ethyl acetate, acetonitrile, dioxane, N,N-dimethylformamide or any other organic solvent which does not adversely influence the reaction.

The reaction is usually carried out at the temperature higher than 100° C., preferably around 140° C. in a sealed tube.

### PROCESS 3

The object compound (I-3) or its salt can be prepared by reacting a compound (IV) or its salt with a compound (III-3) or its salt in a similar manner to the above Process 2.

### PROCESS 4

The object compound (I-4) or its salt can be prepared by reacting a compound M or its salt with a compound (VI) or its salt.

This reaction is usually carried out in the presence of an inorganic or an organic base. Suitable inorganic base and organic base are the same as those exemplified in the above Process 1.

The reaction is usually carried out in a conventional solvent such as an alcohol [e.g., methanol, ethanol, propanol or isopropanol], aromatic hydrocarbon [e.g., benzene, toluene or xylene], ethyl acetate, acetonitrile, dioxane, chloroform, methylene chloride, N,N-dimethylformamide, dimethylsulfoxide or any other organic solvent which does not adversely influence the reaction.

The reaction is usually carried out at the temperature higher than 100° C., preferably around 130° C.

### PROCESS 5

The object compound (I-1) or its salt can be prepared by reacting a compound (VII) or its salt with a compound (III-1) or its salt in a similar manner to the above Process 1 and then treating with hydrochloric acid.

### PROCESS 6

The object compound (I-5) or its salt can be prepared by reacting a compound (VIII) or its salt with a trialkyl orthoformate and an azide compound.

The reaction can be carried out in a conventional organic acid such as acetic acid or propionic acid under heating.

### PROCESS 7

The object compound (I-6) can be prepared by reacting a compound (IX) with a 3-fluorophthalic anhydride and then treating the reaction product with perchloric acid, and then with sulfuric acid.

The reaction can be carried out in a halogenated solvent such as methylene chloride, chloroform, carbon tetrachloride, 1,2-dicholoroethane, at a temperature cooling to heating.

Thus obtained compounds (I-1), (I-2), (I-3), (I-4), (I-5) and (I-6) can be purified by a conventional purification method such as recrystallization, column chromatography, thin-layer chromatography, high-performance liquid chromatography or the like. The compound (I) can be identified by a conventional method such as NMR spectrography, mass spectrography, infrared spectrography, elemental analysis, or measurement of melting point.

Starting compounds (II), (III-1), (III-2), (III-3), (III-4), (IV), (V), (VI), (VII), (VIII) and (IX) are commercially available or can be prepared by the well-known processes, for example, the processes described in M. P. Hay and W. A. Denny, Synthetic Communication, 28(3), 463-470, 1998 or analogous processes thereof.

In order to illustrate the utility of the compound (I), the pharmacological test of the compound (I) is explained in the following.

PARP inhibitory activity (In vitro assay)

(1) Assay method:

The recombinant human PARP (5.3 mg protein/ml) was incubated with a test compound in a 100 μl reaction buffer containing an indicated concentration of 1 mCi/ml ³²P-NAD, 50 mM Tris-HCl, 25 mM MgCl₂, 1 mM DTT (dithiothreitol), 0.05 mM NAD (nicotinamide adenine dinucleotide) and 1 mg/ml activated DNA, pH8.0. Incubation was carried out for 15 minutes at a room temperature, and the reaction was stopped by addition of 200 μl of ice-cold 20% tricholoroacetic acid followed by rapid filtration through GF/B filters. The filtrate was treated with scintillation fluid and acid-insoluble counts were measured for quantification of unit activity. PARP inhibitory activity was calculated by using the following formula: PARP inhibitory activity (%)=[1-(count obtained with test compound)/(count obtained with vehicle only)]×100

(2) Results

TABLE 1
PARP inhibitory activity (IC₅₀) of the test compound.

| Test Compound | IC₅₀(nM) |
|---|---|
| Example 2 | <100 |
| Example 15 | <100 |
| Example 30 | <100 |
| Example 35 | <100 |
| Example 42 | <100 |
| Example 52 | <100 |
| Example 60 | <100 |
| Example 63 | <100 |

The compounds (I) have a potent PARP inhibitory activity as shown in the above. PARP inhibitors of this invention were effective in preventing reduction of striatal DA(dopamine) and its metabolite induced by MPTP (N-methyl-1,2,3,6-tetrahydropyridine) treatment in mice. Therefore, it is suggested that these compounds may have protective benefit in the treatment of neurodegenerative disease such as Parkinson's disease.

It has been known that, during major cellular stresses, the activation of PARP can rapidly lead to cell damage or death through depletion of energy stores and PARP activation play a key role in both NMDA- and NO-induced neurotoxicity (Zhang et. al., Science, 263: 687-89 (1994)). Therefore, the compound (I) of this invention and a pharmaceutically acceptable salt thereof possessing PARP inhibiting activity are useful in treating and preventing various diseases ascribed by NMDA- and NO-induced toxicity. Such diseases include, for example, tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; amyotrophic lateral scleosis (ALS); Huntington's disease; schizophrenia; chronic pain; ischemia and neuronal loss following hypoxia; hypoglycemia; ischemia; trauma; and nervous insult.

It has been demonstrated that PARP inhibitor is useful in reducing infarct size (Thiemermann et al, Proc. Natl. Acad. Sci. USA, 94: 679-83 (1997)). Therefore, the compound (I) of this invention and a pharmaceutically acceptable salt thereof possessing PARP inhibiting activity are useful in treatment and prevention of previously ischemic heart or skeleton muscle tissue.

It is also known that PARP is thought to play a role in enhancing DNA repair. So, the compound (I) of this invention and a pharmaceutically acceptable salt thereof possessing PARP inhibiting activity are effective in treating and preventing radiosensitizing hypoxic tumor cells; tumor cells from recovering from potentially lethal damage of DNA after radiation therapy.

Further, the compound (I) of this invention and a pharmaceutically acceptable salt thereof possessing PARP inhibiting activity are useful in extending the life-span and proliferative capacity of cells and altering gene expression of senescent cells. They are useful for treating and preventing skin aging; Alzheimer's diseases; atheroscleosis; osteoarthritis; osteoporosis; muscular dystrophy; degenerative diseases of skeletal muscle involving replicative senescence; age-related macular degeneration; immune senescence; AIDS; and other immune senescence diseases.

Still further, the compound (I) of this invention and a pharmaceutically acceptable salt thereof possessing PARP inhibiting activity are effective in treating and preventing inflammatory bowel disorders (e.g., colitis); arthritis; diabetes; endotoxic shock; septic shock; and tumor. Also, the compounds (I) are useful in reducing proliferation of tumor cells and making synergistic effect when tumor cells are co-treated with an alkylating drug.

The compound (I) of this invention and a pharmaceutically acceptable salt thereof possessing PARP inhibiting activity are effective in treating and preventing pituitary apoplexy; conjunctivitis; retinoblastoma; retinopathy; acute retinal necrosis syndrome; Sjogren's syndrome.

Accordingly, the present invention provides a method for treating or preventing diseases ascribed by NMDA- and NO-induced toxicity by administering a compound (I), its prodrug, or a pharmaceutically acceptable salt thereof in an effective amount to inhibit PARP activity, to a human being or an animal who needs to be treated or prevented.

The compound (I), its prodrug or their salt can be administered alone or in the form of a mixture, preferably, with a pharmaceutical vehicle or carrier. Accordingly, the present invention provides a pharmaceutical composition comprising a compound (I), its prodrug or a pharmaceutically acceptable salt thereof as an active ingredient in admixture with a pharmaceutically acceptable carrier such as an organic or inorganic carrier or excipient suitable for external (topical), enteral, intravenous, intramuscular, parenteral or intramucous applications in a pharmaceutical preparation, for example, in solid, semisolid or liquid form.

The compound (I), its prodrug or a pharmaceutical acceptable salt thereof can be formulated, for example, with the conventional non-toxic, pharmaceutically acceptable carriers for ointment, cream, plaster, tablets, pellets, capsules, suppositories, solution (saline, for example), emulsion, suspension (olive oil, for example), aerosols, pills, powders, syrup, injection, troches, cataplasms, aromatic water, lotion, buccal tablets, sublingual tablets, nasal drop or any other form suitable for use. The carriers which can be used are water, wax, glucose, lactose, gum acacia, gelatin, mannitol, starch paster, magnesium trisilicate, talc, corn starch, keratin, paraffin, colloidal silica, potato starch, urea and other carriers suitable for use in manufacturing preparations, in solid, semisolid, or liquid form, and in addition to the above auxiliary, stabilizing, thickening or coloring agent and perfume may be used.

The compound (I), its prodrug or a pharmaceutical acceptable salt thereof can be formulated into, for example, preparations for oral application, preparations for injection, preparations for external application, preparations for inhalation, preparations for application to mucous membranes.

The present invention provides a pharmaceutical composition containing a compound (I), its prodrug or a pharmaceutical acceptable salt thereof in admixture of a pharmaceutically acceptable salt for treating or preventing diseases ascribed by NMDA- and NO-induced toxicity, specifically for extending the lifespan or proliferative capacity of cells or altering gene expression of senescent cells, more specifically for treating or preventing diseases ascribed by excess activation of PARP such as tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; Amyotrophic Lateral Scleosis (ALS); Huntington's disease; schizopherenia; chronic pain; ischemia and neuronal loss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; atheroscleosis; osteoarthritis; osteoporosis; muscular dystrophy; degenerative diseases of skeletal muscle involving replicative senescence; age-related macular degeneration; immune senescence; AIDS; and other immune senescencediseases; inflammatory bowel disorders (e.g., colitis); arthritis; diabetes; endotoxic shock; septic shock; and tumor.

Mammals which may be treated by the present invention include livestock mammals such as cows, horses, etc., domestic animals such as dogs, cats, rats, etc. and human beings, preferably human beings.

While the dosage of therapeutically effective amount of the compound (I) varies depending on the age and condition of each individual patient, an average single dose of about 0.01 mg, 0.1 mg, 1 mg, 10 mg, 50 mg, 100 mg, 250 mg, 500 mg, and 1000 mg of the compound (I) may be effective for treating the above-mentioned diseases. In general, amounts between 0.01 mg/body and about 1,000 mg/body may be administered per day.

Any patents, patent applications, and publications cited herein are incorporated by reference.

### BEST MODE FOR CARRYING OUT THE INVENTION

The following Preparation and Examples are given for the purpose of illustrating the present invention in detail, but are not to be construed to limit the scope of the present invention.

Abbreviations used in the following Examples are as follows:
AcOH: acetic acid
DCM: dichloromethane
DMF: N,N-dimethylformamide
EtOAc: ethyl acetate
MeOH: methanol
THF: tetrahydrofuran

### REFERENCE EXAMPLE 1

Under ice cooling, ethyl chloroformate (8.04 g) was added over 30 minutes to a solution of 3-(4-aminophenyl)propanoic acid (10.2 g) in 50% aqueous THF (100 ml) while pH of the solution was maintained between 8 and 10. The solution was stirred for 30 minutes under ice cooling and then sodium chloride (30 g) and EtOAc (50 ml) was added to the solution. The organic layer was separated. The aqueous layer was acidified with 10% aqueous hydrogen chloride and extracted with EtOAc. The combined organic layer was washed with brine, dried over magnesium sulfate and evaporated to give 3-{4-[(ethoxycarbonyl)amino]phenyl}-propanoic acid (10.2 g). ¹H-NMR (DMSO-d₆) δ: 1.23(3H, t, J=7.1 Hz), 2.4-2.6(2H, m), 2.7-2.8(2H, m), 4.10(2H, q, J=7.1 Hz), 7.07(2H, d, J=8.5 Hz), 7.34(2H, d, J=8.5 Hz), 9.49(1H, s).

Mass: 236.27 (M-H)−.

### REFERENCE EXAMPLE 2

Ethyl 4-(4-hydroxybutyl)phenylcarbamate was obtained in a similar manner to Reference Example 1.

¹H-NMR (DMSO-d₆) δ: 1.23(3H, t, J=7.1 Hz), 1.35-1.65(4H, m), 2.45-2.55(2H, m), 3.3-3.45(2H, m), 4.10(2H, q, J=7.1 Hz), 4.33(1H, t, J=5.2 Hz), 7.07(2H, d, J=8.5 Hz), 7.34(2H, d, J=8.5 Hz), 9.46(1H, s)

Mass: 260.2 (M+Na)+.

### REFERENCE EXAMPLE 3

Bromine (3.51 g) was added to a solution of ethyl 4-(3-hydroxypropyl)phenylcarbamate (4.46 g) and sodium acetate (3.28 g) in AcOH (50 ml), and the mixture was stirred for 5 hours. After evaporation of the solvent, the residue was diluted with a mixture of water and EtOAc. The separated organic layer was washed with an aqueous saturated sodium hydrogencarbonate solution, an aqueous sodium thiosulfate solution and brine, successively and dried over magnesium sulfate. After evaporation of the solvent, the residue was purified by column chromatography on silica-gel eluting with a mixture of n-hexane and EtOAc to give ethyl 2-bromo-4-(3-hydroxypropyl)phenylcarbamate (5.53 g).

¹H-NMR (DMSO-d₆) δ:1.32(3H, t, J=7.1 Hz), 1.8-2.0(2H, m), 2.65(2H, t, J=7.2 Hz), 3.6-3.7(2H, m), 4.23(2H, q, J=7.1 Hz), 7.02(1H, br s), 7.13(1H, dd, J=8.4, 2.0 Hz), 7.35(1H, d, J=2.0 Hz), 8.01(1H, d, J=8.4 Hz).

Mass: 303.67 (M+H)+.

### REFERENCE EXAMPLE 4

Ethyl 2-bromo-4-(4-hydroxybutyl)phenylcarbamate was obtained in a similar manner to Reference Example 3. ¹H-NMR (CDCl₃) δ: 1.32(3H, t, J=7.1 Hz), 1.4-1.8(5H, m), 2.58(2H, t, J=7.1 Hz), 3.65(2H, t, J=6.3 Hz), 4.24(2H, q, J=7.1 Hz), 7.01(1H, s), 7.11(1H, dd, J=8.4, 2.0 Hz), 7.33(1H, d, J=2.0 Hz), 8.00(1H, d, J=8.4 Hz).

Mass: 338.1, 340.1 (M+Na)+.

### REFERENCE EXAMPLE 5

Under a nitrogen atmosphere, phosphorus tribromide (0.57 ml) was added to a solution of ethyl 2-bromo-4-(3-hydroxypropyl)phenyl-carbamate (5.2 g) in EtOAc (50 ml) at −20° C. The mixture was stirred for 1 hour under ice cooling. After the ice bath was removed, the mixture was stirred overnight at ambient temperature. The mixture was poured into a mixture of an aqueous saturated sodium hydrogen carbonate solution and EtOAc. The separated organic layer was washed with brine and dried over magnesium sulfate. After evaporation of the solvent, the residue was purified by column chromatography on silica-gel eluting with a mixture of n-hexane and EtOAc to give ethyl

2-bromo-4-(3-bromopropyl)phenylcarbamate (4.1 g). ¹H-NMR (CDCl₃) δ: 1.33(3H, t, J=7.1 Hz), 2.0-2.0(2H, m), 2.65-2.8(2H, m), 3.37(2H, t, J=6.5 Hz), 4.24(2H, q, J=7.1 Hz), 7.03(1H, br s), 7.13(1H, dd, J=8.4, 2.0 Hz), 7.36(1H, d, J=2.0 Hz), 8.04(1H, d, J=8.4 Hz).

Mass: 388.0 (M+Na)+.

### REFERENCE EXAMPLE 6

The following compounds (1) and (2) were obtained in a similar manner to Reference Example 5.

(1)

Ethyl 2-bromo-4-(4-bromobutyl)phenylcarbamate ¹H-NMR (CDCl₃) δ: 1.33(3H, t, J=7.1 Hz), 1.65-2.0(4H, m), 2.57(2H, t, J=7.1 Hz), 3.41(2H, t, J=6.1 Hz), 4.24(2H, q, J=7.1 Hz), 7.02(1H, br s), 7.11(1H, dd, J=8.2, 2.0 Hz), 7.32(1H, d, J=2.0 Hz), 8.02(1H, d, J=8.4 Hz).

Mass: 400.0, 402.0 (M+Na)+.

(2)

N-[3-(Bromomethyl)phenyl)-1,4-dioxaspiro[4.5]decane-6-carboxamide

¹H-NMR (DMSO-d₆) δ: 1.2-2.0(8H, m), 2.6-2.7(1H, m), 3.7-4.1(4H, m), 4.52(2H, s), 6.97(1H, d, J=7.8 Hz), 7.24(1H, t, J=7.8 Hz), 7.43(1H, d, J=7.8 Hz), 7.76(1H, s), 9.72(1H, s).

### REFERENCE EXAMPLE 7

Under a nitrogen atmosphere, phenylboronic acid (437 mg), 2M aqueous solution of sodium dicarbonate (4.5 ml) and tetrakis(triphenylphosphine)palladium (0) (173 mg) were added to a solution of ethyl 2-bromo-4-(3-bromopropyl)phenylcarbamate (1.1 g) in dimethoxyethane (13.5 ml) at room temperature. The mixture was refluxed for 5 hours. After cooling to room temperature, the mitre was poured into a mixture of water and EtOAc. The separated organic layer was washed with brine and dried over magnesium sulfate. After evaporation of the solvent, the residue was purified by column chromatography on silica-gel eluting with toluene to give ethyl 5-(3-bromopropyl)-1,1′-biphenyl-2-ylcarbamate (1.1 g).

¹H-NMR (CDCl₃) δ: 1.24(3H, t, J=7.1 Hz), 2.0-2.4(2H, m), 2.76(2H, t, J=7.0 Hz), 3.40(2H, t, J=6.6 Hz), 4.16(2H, q, J=7.1 Hz), 6.55(1H, br s), 7.0-7.5(7H, m), 8.02(1H, d, J=8.3 Hz).

Mass: 384.1, 386.1 (M+Na)+.

### REFERENCE EXAMPLE 8

The following compounds described in (1) and (2) were obtained in a similar manner to Reference Example 7.

(1)

Ethyl 5-(3-bromopropyl)-4′-chloro-1,1′-biphenyl-2-ylcarbamate ¹H-NMR (CDCl₃) δ: 1.25(3H, t, J=7.1 Hz), 2.0-2.3(2H, m), 2.76(2H, t, J=7.0 Hz), 3.3-3.5(2H, m), 4.16(2H, q, J=7.1 Hz), 6.41(1H, br s), 6.7-7.5(6H, m), 7.98(1H, d, J=8.0 Hz).

Mass: 418.1, 420.1 (M+Na)+.

(2)

Ethyl 5-(4-bromobutyl)-1,1′-biphenyl-2-ylcarbamate ¹H-NMR (CDCl₃) δ: 1.24(3H, t, J=7.2 Hz), 1.65-2.0(4H, m), 2.55-2.75(2H, m), 3.41(2H, q, J=7.0 Hz), 4.16(2H, q, J=7.2 Hz), 6.53(1H, br s), 7.0-7.5(7H, m), 8.01(1H, d, J=8.3 Hz).

Mass: 398.1, 400.2 (M+H)+.

### REFERENCE EXAMPLE 9

Under a nitrogen atmosphere, phosphorus pentoxide (511 mg) was added to a solution of ethyl 5-(3-bromopropyl)-1,1′-biphenyl-2-ylcarbamate (435 mg) in phosphorus oxychloride (3 ml) at room temperature. The mire was refluxed for 2 hours. After evaporation of the solvent, the residue was poured into a mixture of ice-water and EtOAc. The solution was brought to pH 9 with 10% aqueous solution of potassium carbonate. The separated organic layer was washed with brine and dried over magnesium sulfate. After evaporation of the solvent in vacuo, the residue was dissolved in a mixture of dioxane (6 ml) and 4N aqueous hydrogen chloride (3 ml). The solution was refluxed for 30 minutes, cooled to room temperature and then poured into a mixture of water and EtOAc. The mixture was neutralized with 10% aqueous solution of potassium carbonate. The separated organic layer was washed with brine and dried over magnesium sulfate. After evaporation of the solvent, the residue was purified by column chromatography on silica-gel eluting with a mixture of DCM and acetone to give 2-(3-bromopropyl)-6(5H)-phenanthridinone (280 mg).

¹H-NMR (DMSO-d₆) δ: 2.0-2.3(2H, m), 2.83(2H, t, J=7.0 Hz), 3.5-3.7(2H, m), 7.25-7.4(2H, m), 7.63(1H, t, J=7.1 Hz), 7.85(1H, dt, J=7.2, 1.5 Hz), 8.23(1H, s), 8.32(1H, dt, J=7.9, 1.2 Hz), 8.52(1H, d, J=8.1 Hz), 11.62(1H, s).

Mass: 316.2, 318.2 (M+H)+.

### REFERENCE EXAMPLE 10

The following compounds described in (1) and (2) were obtained in a similar manner to Reference Example 9.

(1)

2-(3-Bromopropyl)-8-chloro-6(5H)-phenanthridinone ¹H-NMR (DMSO-d₆) δ: 2.0-2.4(2H, m), 2.7-2.9(2H, m), 3.4-3.8(2H, m), 7.2-7.5(3H, m), 7.8-7.95(1H, m), 8.2-8.3(2H, m), 8.57(1H, d, J=8.8 Hz), 11.79(1H, s).

Mass: 372.1, 374.1 (M+Na)+.

(2)

2-(4-Chlorobutyl)-6 (5H)-phenanthridinone ¹H-NMR (DMSO-d₆) δ: 1.7-2.0(4H, m), 2.65-2.85(2H, m), 3.6-3.75(2H, m), 7.25-7.35(2H, m), 7.55-7.7(1H, m), 7.8-7.9(1H, m), 8.21(1H, s), 8.3-8.4(1H, m), 8.52(1H, d, J=8.3 Hz), 11.61(1H, s).

Mass: 308.3 (M+Na)+.

### REFERENCE EXAMPLE 11

A mixture of 50% Pd/C catalyst (50% wet, 2.72 g) and 1-(4-hydroxybutyl)-4-nitrobenzene (5 g) in MeOH (50 ml) was stirred under hydrogen at atmospheric pressure until hydrogen gas absorption stopped. After filtration of the reaction mixture on celite, the filtrate was concentrated in vacuo to give 4-(4-hydroxybutyl)aniline (4.0 g). ¹H-NMR (DMSO-d₆) δ: 1.3-1.6(4H, m), 2.38(2H, t, J=7.1 Hz), 3.3-3.45(2H, m), 4.31(1H, t, J=5.2 Hz), 4.77(2H, s), 6.4-6.55(2H, m), 6.75-6.9(2H, m).

Mass: 166.4 (M+H)+.

### REFERENCE EXAMPLE 12

Under a nitrogen atmosphere, 4-nitrophenol (6.95 g) was added portionwise to a solution of potassium tert-butoxide (6.73 g) in DMF (70 ml) with ice cooling. After the mire was stirred for 5 minutes, bromochloroethane (7.88 g) was added to the mixture. The mixture was stirred at ambient temperature for 30 minutes and then heated at 80° C. for 4 hours. The mixture was cooled to room temperature and poured into a mixture of water and EtOAc. The separated organic layer was washed with water and brine, successively and dried over magnesium sulfate. After evaporation of the solvent, the residue was purified by column chromatography on silica-gel eluting with a mixture of n-hexane and EtOAc to give 1-(2-chloroethoxy)-4-nitrobenzene (4.37 g).

¹H-NMR (CDCl₃) δ: 3.85(2H, t, J=5.7 Hz), 4.33(2H, t, J=5.7 Hz), 6.9-7.0(2H, m), 8.15-8.25(2H, m).

### REFERENCE EXAMPLE 13

Ammonium chloride (430 mg) was added to a mixture of 1-(2-chloroethoxy)-4-nitrobenzene (4.3 g) in THF (40 ml), ethanol (80 ml) and water (12 ml). The mixture was gradually warmed to 50° C. and iron (reduced) (4.3 g) was added portionwise thereto. The whole mixture was refluxed for 1 hour and then cooled to room temperature. After unsolvable material was removed by filtration on celite, the filtrate was concentrated in vacuo. The residue was diluted with EtOAc and the obtained solution was washed with water and brine, successively. After the solution was dried over magnesium sulfate, the solution was evaporated to give 4-(2-chloroethoxy)aniline (2.7 g). ¹H-NMR (CDCl₃) δ: 3.76(2H, t, J=5.9 Hz), 4.15(2H, t, J=5.9 Hz), 6.5-6.85(4H, m).

### REFERENCE EXAMPLE 14

3-(2-Bromoethyl)aniline hydrochloride was obtained in a similar manner to Reference Example 13.

¹H-NMR (DMSO-d₆) δ: 3.16(2H, t, J=7.0 Hz), 3.74(2H, t, J=7.0 Hz), 7.15-7.45(4H, m).

Mass: 200.1, 202.2(M+H)+.

### REFERENCE EXAMPLE 15

4-(2-Chloroethoxy)aniline (1.72 g) was added to a solution of ethyl 2-cyclohexanonecarboxylate (2.3 g) in xylene (4 ml). The mixture was heated at 190° C. for 1 hour and then cooled to room temperature. The solution was poured into a mixture of water and EtOAc The separated organic layer was washed with brine and dried over magnesium sulfate. After evaporation of the solvent, the residue was dissolved in 90% sulfuric acid (8 ml). The solution was heated at 60° C. for 30 minutes, poured on ice and then stirred for 30 minutes. The resulting precipitate was collected by filtration and dissolved in EtOAc. The organic solution was washed with water and brine, successively and dried over magnesium sulfate. After evaporation of the solvent, the residue was purified by column chromatography on silica-gel eluting with a mixture of DCM and acetone to give 2-(2-chloroethoxy)-7,8,9,10-tetrahydro-6(5H)-phenanthridinone (220 mg).

¹H-NMR (DMSO-d₆) δ: 1.6-1.9(4H, m), 2.4-2.6(2H, m), 2.7-2.8(2H, m), 3.9-4.0(2H, m), 4.25-4.35(2H, m), 7.05-7.25(3H, m), 11.50(1H, s).

Mass: 300.1, 302.1(M+Na)+.

### REFERENCE EXAMPLE 16

Under ice cooling, 10N THF solution of borane-methyl sulfide complex (2.35 ml) was added slowly to a solution of 3-{4-[(tert-butoxycarbonyl)amino]phenyl}propanoic acid (5.2 g) in THF (50 ml). The ice bath was removed after 5 minutes of the addition. The mixture was stirred at ambient temperature for 1 hour. After the reaction was quenched with water, the mixture was poured into a mixture of cold water and EtOAc. The mixture was brought to be basic with an aqueous saturated sodium hydrogencarbonate solution. The separated organic layer was washed with brine and dried over magnesium sulfate. After evaporation of the solvent, the residue was purified by column chromatography on silica-gel eluting with a mixture of DCM and acetone to give tert-butyl 4-(3-hydroxypropyl)phenylcarbamate (4.6 g).

¹H-NMR (DMSO-d₆) δ: 1.46(9H, s), 1.6-1.8(2H, m), 2.45-2.65(2H, m), 3.40(2H, q, J=6.4 Hz), 4.44(1H, t, J=5.2 Hz), 6.78(1H, d, J=7.2 Hz), 7.12(1H, t, J=7.2 Hz), 7.21(1H, d, J=7.2 Hz), 7.33(1H, s), 9.22(1H, s).

Mass: 274.3(M+Na)+.

### REFERENCE EXAMPLE 17

Ethyl 4-(3-hydroxypropyl)phenyl carbamate was obtained in a similar manner to Reference Example 16.

¹H-NMR (CDCl₃) δ: 1.30(3H, t, J=7.1 Hz), 1.8-1.95(2H, m), 2.66(2H, t, J=7.2 Hz), 3.6-3.7(2H, m), 4.21(2H, q, J=7.1 Hz), 6.60(1H, br s), 7.12(2H, d, J=8.6 Hz), 7.28(2H, d, J=8.6 Hz).

Mass: 246.3 (M+Na)+.

### REFERENCE EXAMPLE 18

Under a nitrogen atmosphere, triethylamine (7.7 ml) and methanesulfonyl chloride (1.6 ml) were added successively to a solution of tert-butyl 4-(3-hydroxypropyl)phenylcarbamate (4.6 g) in DCM (50 ml) at −15° C. The mixture was stirred for 1 hour at the same temperature and then poured into a mixture of water and EtOAc. The separated organic layer was washed with diluted aqueous hydrogen chloride and brine, successively and dried over magnesium sulfate. The organic layer was evaporated under reduced pressure to give 3-{4-[(tert-butoxycarbonyl)amino]phenyl}propyl methanesulfonate (6.5 g).

¹H-NMR (DMSO-d₆) δ: 1.46(9H, s), 1.9-2.0(2H, m), 3.61(2H, t, J=6.4 Hz), 3.15(3H, s), 4.19(2H, t, J=6.4 Hz), 6.82(1H, d, J=7.2 Hz), 7.1-7.3(2H, m), 7.36(1H, s), 9.26(1H, s).

Mass: 328.2(M-H)−

### REFERENCE EXAMPLE 19

Under a nitrogen atmosphere, sodium bromide (4.09 g) was added to a solution of 3-{4-[(tert-butoxycarbonyl)amino]phenyl}propyl methanesulfonate (6.54 g) in DMF (60 ml) at room temperature. The mixture was stirred for 2 hours at 60° C. and poured into a mixture of water and EtOAc. The separated organic layer was washed twice with water and brine, successively and dried over magnesium sulfate. The organic layer was evaporated to give tert-butyl 4-(3-bromopropyl)phenylcarbamate (5.30 g).

¹H-NMR (DMSO-d₆) δ: 1.46(9H, s), 2.0-2.2(2H, m), 2.5-2.7(2H, m), 3.50(2H, t, J=6.6 Hz), 6.80(1H, d, J=7.3 Hz), 7.15(1H, t, J=7.3 Hz), 7.25(1H, d, J=7.3 Hz), 7.35(1H, s), 9.26(1H, s).

Mass: 336.1, 338.2(M+Na)+

### REFERENCE EXAMPLE 20

Trifluoroacetic acid (13 ml) was added to a solution of tert-butyl 4-(3-bromopropyl)phenylcarbamate (5.25 g) in DCM at room temperature. The mixture was stirred for 4 hours. After evaporation of the solvent, diethyl ether was added to the residue to wash the crude product. After the ethereal layer was removed by decantation, the resulting crude oil was diluted with EtOAc. After adding 4N hydrogen chloride in EtOAc (10 ml) to the solution, the resulting precipitate was collected by filtration, washed with EtOAc and dried in vacuo to give 3-(3-bromopropyl)aniline hydrochloride (2.32 g).

¹H-NMR (DMSO-d₆) δ: 1.95-2.20(2H, m), 2.5-2.8(2H, m), 3.52(2H, t, J=6.6 Hz), 7.15-7.30(2H, m), 7.35-7.50(1H, m).

Mass: 214.2, 216.1(M+H)+.

### REFERENCE EXAMPLE 21

Oxalyl chloride (1.14 g) was added dropwise to a solution of 1,4-dioxaspiro[4,5]decane-6-carboxylic acid (559 mg) and DMF (1 drop) in DCM (5 ml), and the mixture was stirred for 2 hours at room temperature. After removing the solvent under reduced pressure, the residue was dissolved in DCM (5 ml). The solution was added dropwise to a solution of 3-(3-bromopropyl)aniline hydrochloride (752 mg) and triethylamine (1.67 ml) in DCM (10 ml). The solution was stirred for 2 hours at room temperature and poured into a mixture of water and DCM. The separated organic layer was washed with 1N aqueous hydrogen chloride, water, an aqueous saturated sodium hydrogencarbonate solution and brine, successively and dried over magnesium sulfate. After evaporation of the solvent, the residue was purified by column chromatography on silica-gel eluting with a mixture of DCM and acetone to give

N-[3-(3-bromopropyl)phenyl]-1,4-dioxaspiro[4.5]decane-6-carboxamide (1.07 g).

¹H-NMR (DMSO-d₆) δ: 1.2-2.2(10H, m), 2.6-2.7(3H, m), 3.51(2H, t, J=6.6 Hz), 3.75-3.90(4H, m), 6.87(1H, d, J=7.7 Hz), 7.19(1H, t, J=7.7 Hz), 7.41(1H, d, J=7.7 Hz), 7.48(1H, s), 9.57(1H, s).

Mass: 380.1, 382.2(M-H)−

### REFERENCE EXAMPLE 22

The following compounds (1) to (4) were obtained in a similar manner to Reference Example 21.

(1)

N-[3-(2-Bromoethyl)phenyl]-1,4-dioxaspiro[4.5]decane-6-carboxamide

¹H-NMR (DMSO-d₆) δ: 1.2-2.0(8H, m), 2.6-2.7(1H, m), 3.08(2H, t, J=7.1 Hz), 3.70(2H, t, J=7.1 Hz), 3.7-3.9(4H, m), 6.94(1H, d, J=7.6 Hz), 7.21(1H, t, J=7.6 Hz), 7.45(1H, d, J=7.6 Hz), 7.50(1H, s), 9.59(1H, s).

Mass: 390.1, 392.1 (M+Na)+.

(2)

N-(3-Bromophenyl)-1,4-dioxaspiro[4.5]decane-6-carboxamide ¹H-NMR (DMSO-d₆) δ: 1.2-2.0(8H, m), 2.6-2.7(1H, m), 3.7-3.9(4H, m), 7.15-7.30(2H, m), 7.4-7.5(1H, m), 7.99(1H, s), 9.83(1H, s).

Mass: 338.1, 340.1 (M-H)−.

(3)

N-[3-(Methylthio)phenyl]-1,4-dioxaspiro[4.5]decane-6-carboxamide ¹H-NMR (DMSO-d₆) δ: 1.2-1.95(6H, m), 2.44(3H, s), 2.6-2.65(1H, m), 3.75-3.90(4H, m), 6.85-6.95(1H, m), 7.21(1H, t, J=7.9 Hz), 7.31(1H, d, J=7.9 Hz), 7.60(1H, s), 9.66(1H, s).

Mass: 330.3(M+Na)+.

(4)

N-(2-Methoxyphenyl)-1,4-dioxaspiro[4.5]decane-6-carboxamide ¹H-NMR (DMSO-d₆) δ: 1.25-1.8(6H, m), 1.85-1.95(2H, m), 2.70-2.75(1H, m), 3.86(3H, s), 3.9-4.0(4H, m), 6.85-7.05(4H, m), 8.16(1H, d, J=7.6 Hz), 9.15(1H, s).

Mass: 314.3(M+Na)+.

### REFERENCE EXAMPLE 23

60% Perchloric acid (1.35 g) was added to a solution of N-[3-(3-bromopropyl)phenyl]-1,4-dioxaspiro[4.5]decane-6-carboxamide (1.03 g) in DCM (10 ml) at room temperature and the mixture was stirred for 10 minutes. The solution was carefully poured into an aqueous saturated sodium hydrogencarbonate solution and the mixture was stirred for 30 minutes. The organic layer was separated and the aqueous layer was extracted with chloroform. The combined organic layer was dried over magnesium sulfate. After evaporation of the solvent, the residue was dissolved in 90% aqueous sulfonic acid. The solution was heated at 60° C. for 20 minutes and then poured on ice. The solution was stirred for 30 minutes. The resulting precipitate was collected by filtration, washed successfully with water and dried in vacuo to give 3-(3-bromopropyl)-7,8,9,10-tetrahydro-6(5H)-phenanthridinone (580 mg).

¹H-NMR (DMSO-d₆) δ: 1.60-1.75(4H, m), 2.0-2.2(2H, m), 2.4-2.5(2H, m), 2.7-2.8(2H, m), 3.52(2H, t, J=6.6 Hz), 7.04(1H, d, J=8.3 Hz), 7.10(1H, s), 7.59(1H, d, J=8.3 Hz), 11.52(1H, s).

Mass: 318.2, 320.1 (M+H)+

### REFERENCE EXAMPLE 24

The following compounds (1) to (5) were obtained in a similar manner to Reference Example 23.

(1)

3-(2-Bromoethyl)-7,8,9,10-tetrahydro-6(5H)-phenanthridinone 1H-NMR (DMSO-d₆) δ: 1.6-1.9(4H, m), 2.45-2.55(2H, m), 2.75-2.90(2H, m), 3.17(2H, t, J=7.1 Hz), 3.74(2H, t, J=7.1 Hz), 7.10(1H, d, J=8.2 Hz), 7.12(1H, s), 7.62(1H, d, J=8.2 Hz).

Mass: 328.2, 330.1(M+Na)+.

(2)

3-Bromo-7,8,9,10-tetrahydro-6 (5H)-phenanthridinone ¹H-NMR (DMSO-d₆) δ: 1.6-1.75(4H, m), 2.4-2.5(2H, m), 2.7-2.8(2H, m), 7.32(1H, dd, J=8.6, 1.9 Hz), 7.45(1H, d, J=1.9 Hz), 7.61(1H, d, J=8.6 Hz), 11.67(1H, s).

Mass: 300.1, 302.1(M+Na)+.

(3)

3-(Methylthio)-7,8,9,10-tetrahydro-6(5H)-phenanthridinone ¹H-NMR (DMSO-d₆) δ: 1.65-1.85(4H, m), 2.4-2.5(2H, m), 2.75-2.85(2H, m), 3.45(3H, s), 7.05(1H, d, J=8.3 Hz), 7.11(1H, s), 7.58(1H, d, J=8.3 Hz), 11.48(1H, s).

Mass: 258.2(M+Na)+.

(4)

4-Methoxy-7,8,9,10-tetrahydro-6(5H)-phenanthridinone ¹H-NMR (DMSO-d₆) δ: 1.65-1.85(4H, m), 2.4-2.5(2H, m), 2.75-2.85(2H, m), 3.89(3H, s), 7.05-7.15(2H, m), 7.25-7.30(1H, m), 10.51(1H, s).

Mass: 252.3(M+Na)+.

(5)

3-(Bromomethyl)-7,8,9,10-tetrahydro-6(5H)-phenanthridinone ¹H-NMR (DMSO-d₆) δ: 1.6-1.9(4H, m), 2.4-2.55(2H, m), 2.75-2.9(2H, m), 3.56(2H, s), 7.23(1H, dd, J=8.3, 1.6 Hz), 7.32(1H, d, J=1.6 Hz), 7.66(1H, d, J=8.3 Hz), 11.67(1H, s).

Mass: 314.1, 316.0 (M+Na)+.

### REFERENCE EXAMPLE 25

A suspension of 3-(methylthio)-7,8,9,10-tetrahydro-6(5H)-phenanthridinone (180 mg) in DMF (18 ml) was heated at 90° C. to solve the compound. OXONE® (monopersulfate compound, 2KHSO₅·KHSO₄·K₂SO₄, produced by Du Pont) (902 mg) in water (3 ml) was added to this solution. The mixture was stirred for 30 minutes at the same temperature and stirred overnight at room temperature. The mixture was poured into a mixture of water and EtOAc. The separated organic layer was washed with brine and dried over magnesium sulfate. After evaporation of the solvent, the residue was recrystallized in MeOH. The crystalline was collected by filtration, washed with MeOH and dried under reduced pressure to give 3-(methylsulfonyl)-7,8,9,10-tetrahydro-6(5H)-phenanthridinone (112 mg).

IR (KBr) cm⁻¹: 2931, 1660, 1641, 1560.

¹H-NMR (DMSO-d₆) δ: 1.65-1.85(4H, m), 2.45-2.55(2H, m), 2.8-2.9(2H, m), 3.24(3H, s), 7.66(1H, dd, J=8.5, 1.8 Hz), 7.82(1H, d, J=1.8 Hz), 7.91(1H, d, J=8.5 Hz), 11.95(1H, s).

### REFERENCE EXAMPLE 26

Under a nitrogen atmosphere, 1M DCM solution of boron tribromide (4.4 ml) was added to a solution of 4-methoxy-7,8,9,10-tetrahydro-6(5H)-phenanthridinone (252 mg) in DCM (10 ml) at 0° C. The mixture was stirred for 2 hours and poured into a mixture of water and EtOAc. The separated organic layer was washed with brine and dried over magnesium sulfate. After evaporation of the solvent, the crude product was recrystallized in MeOH. The crystalline was collected by filtration, washed with MeOH and dried under reduced pressure to give 4-hydroxy-7,8,9,10-tetrahydro-6(5H)-phenanthridinone (151 mg).

IR (KBr) cm⁻¹: 1644, 1602, 1563.

¹H-NMR (DMSO-d₆) δ: 1.6-1.85(4H, m), 2.35-2.45(2H, m), 2.7-2.9(2H, m), 6.91(1H, dd, J=7.6, 1.2 Hz), 6.99(1H, t, J=7.6 Hz), 7.14(1H, dd, J=7.6, 1.2 Hz), 10.15(1H, s).

Mass: 238.2(M+Na)+.

### REFERENCE EXAMPLE 27

To a solution of 1,4-dioxaspiro[4,5]decane-6-carboxylic acid (1.87 g) and 3-aminobenzylalcohol (1.24 g) in DCM (100 ml) were added successively 1-(3-dimethylaminopropyl)-3-ethylcarbodiimide hydrochloride (2.89 g) and N,N-dimethylaminopyridine (613 mg). The mixture was stirred overnight at room temperature and poured into a mixture of water and DCM. The separated organic layer was washed with a diluted aqueous hydrogen chloride solution and brine, successively and dried over magnesium sulfate. After evaporation of the solvent, the residue was purified by column chromatography on silica-gel eluting with a mixture of DCM and acetone to give N-[3-(hydroxymethyl)phenyl)-1,4-dioxaspiro[4.5]decane-6-carboxamide (1.61 g).

1H-NMR (DMSO-d₆) δ: 1.2-2.0(8H, m), 2.6-2.7(1H, m), 3.7-4.0(4H, m), 4.44(2H, d, J=5.7 Hz), 5.16(1H, t, J=5.7 Hz), 6.95(1H, d, J=7.8 Hz), 7.20(1H, t, J=7.8 Hz), 7.44(1H, d, J=7.8 Hz), 7.58(1H, s), 9.60(1H, s).

### REFERENCE EXAMPLE 28

Oxaryl chloride (3.82 g) and 1 drop of DMF were added successively to a solution of 1,4-dioxaspiro[4,5]decane-6-carboxylic acid (1.87 g) in DCM (15 ml) at room temperature. The solution was stirred for 2 hours at room temperature and the solvent was evaporated. The residue was diluted with DCM (5 ml) and added dropwise to a mixture of 3-nitroaniline (1.39 g) and triethylamine (3.05 g) in DCM (8.5 ml) under ice cooling. After 10 minutes the ice bath was removed and the mixture was stirred at room temperature for 1.5 hours and poured into a mixture of water and EtOAc. The organic phase was separated and washed with diluted aqueous hydrogen chloride, brine and then dried over magnesium sulfate. After evaporation of the solvent, the residue was purified by column chromatography on silica-gel eluting with DCM-acetone to afford N-(3-nitrophenyl)-1,4-dioxaspiro[4.5]decane-6-carboxamide (1.6 g).

¹H NMR (DMSO-d₆) δ: 1.2-2.0(8H, m), 2.6-2.75(1H, m), 3.7-3.95(4H, m), 7.58(1H, t, J=8.1 Hz), 7.8-8.0(2H, m), 8.68(1H, t, J=2.1 Hz), 10.20(1H, s).

Mass (APCI) m/e:329.2(M+Na)+.

### REFERENCE EXAMPLE 29

10% Palladium on carbon (50% wet, 160 mg) was added to a solution of N-(3-nitrophenyl)-1,4-dioxaspiro[4,5]decane-6-carboxamide (1.6 g) in MeOH (20 ml). The mixture was hydrogenated under hydrogen atmosphere at atmospheric pressure for 6 hours. Unsoluble material was removed by filtration through celite. The filtrate was concentrated in vacuo to afford N-(3-aminophenyl)-1,4-dioxaspiro[4,5]decane-6-carboxamide (1.24 g). ¹H NMR (DMSO-d₆) δ: 1.2-2.0(8H, m), 2.63(1H, dd, J=11.1, 4.6 Hz), 3.9-4.05(4H, m), 6.35-6.45(1H, m), 6.43(1H, dd, J=7.8, 1.5 Hz), 7.05(1H, t, J=7.8 Hz), 7.2-7.3(1H, m), 8.22(1H, s).

Mass (APCI) m/e: 299.3(M+Na)+.

### REFERENCE EXAMPLE 30

N-(3-aminophenyl)-1,4-dioxaspiro[4,5]decane-6-carboxamide (930 mg) was dissolved in chloroform (15 ml), and phthalic anhydride (499 mg) was added to the solution. The mixture was stirred under reflux for 4 hours and cooled to room temperature. The solvent was evaporated in vacuo and the resulting residue was purified by column chromatography on silica-gel eluting with hexane-EtOAc to afford N-[3-(1,3-dioxo-1,3-dihydro-2H-isoindol-2-yl)phenyl]-1,4-dioxaspiro-[4, 5]decane-6-carboxamide (800 mg).

¹H NMR (DMSO-d₆) δ: 1.1-2.0(8H, m), 2.6-2.7(1H, m), 3.75-3.95(4H, m), 7.10(1H, dd, J=8.0, 1.8 Hz), 7.42(1H, t, J=8.0 Hz), 7.61(1H, d, J=8.0 Hz), 7.77(1H, t, J=1.8 Hz), 7.85-8.0(4H, m), 9.89(1H, s).

Mass (APCI) m/e: 429.2(M+Na)+.

### REFERENCE EXAMPLE 31

N-[3-(4-fluoro-1,3-dioxo-1,3-dihydro-2H-isoindol-2-yl)-phenyl]-1,4-dioxaspiro[4,5]decane-6-carboxamide was obtained in a similar manner to Reference Example 30.

¹H NMR (DMSO-d₆) δ: 1.2-2.0(8H, m), 2.6-2.8(1H, m), 3.75-4.0(4H, m), 7.12(1H, d, J=8.0 Hz), 7.43(1H, t, J=8.0 Hz), 7.45-8.0(4H, m), 9.89(1H, s).

### REFERENCE EXAMPLE 32

60% Perchloric acid (1.06 g) was added to a solution of N-13-(1,3-dioxo-1,3-dihydro-2H-isoindol-2-yl)phenyl]-1,4-dioxaspiro-[4,5]decane-6-carboxamide (860 mg) in DCM (50 ml) at room temperature and stirred for 10 minutes. The solution was carefully poured into saturated aqueous solution of sodium hydrogencarbonate and stirred for 30 minutes. The organic layer was dried over magnesium sulfate. After evaporation of the solvents, the residue was dissolved in 90% sulfuric acid. The solution was heated at 60° C. for 20 minutes and poured on ice. The solution was stirred for 30 minutes and the resulting precipitates were collected by filtration, washed with water and dried in vacuo to afford 2-(6-oxo-5,6,7,8,9,10-hexahydro-3-phenanthridinyl)-1H-isoindol-1,3(2H)-dione (480 mg).

¹H NMR (DMSO-d₆) δ: 1.6-1.8(6H, m), 2.8-3.0(2H, m), 7.28(1H, dd, J=8.7, 1.9 Hz), 7.40(1H, d, J=1.9 Hz), 7.81(1H, d, J=8.7 Hz), 7.85-8.0(4H, m), 11.80(1H, s).

Mass (APCI m/e: 367.2(M+Na)+.

### REFERENCE EXAMPLE 33

Hydrazine monohydrate (209 mg) was added to a solution of 2-(6-oxo-5,6,7,8,9,10-hexahydro-3-phenanthridinyl)-3a,7a-dihydro-1H-isoindol-1,3(2H)-dione (480 mg) in THF (20 ml). The mixture was stirred under reflux for 9 hours and cooled to room temperature. The solvent was evaporated in vacuo and the residue was purified by column chromatography on silica-gel eluting with DCM-acetone to afford 3-amino-7,8,9,10-tetrahydro-6(5H)-phenanthridinone (280 mg).

¹H NMR (DMSO-d₆) δ: 1.7-1.9(4H, m), 2.3-2.45(2H, m), 2.6-2.8(2H, m), 5.5(2H, br s), 6.36(1H, d, J=2.1 Hz), 6.44(1H, dd, J=8.6, 2.1 Hz), 7.31(1H, d, J=8.6 Hz), 11.16(1H, s).

Mass (APCI) m/e: 237.3(M+Na)+.

### REFERENCE EXAMPLE 34

Copper (1.95 g) was added to a mixture of methyl 2-iodobenzoate (7.0 g) and 4-bromo-3-nitrobenzoic acid methylester (6.95 g). The whole mixture was stirred at 200° C. for 5 hours. The mixture was cooled to room temperature and diluted with a mixture of EtOAc and water. Copper was removed by filtration, and the organic phase was separated, washed with water and brine and then dried over magnesium sulfate. After evaporation of the solvent the residue was purified by column chromatography on silica-gel eluting with hexane-EtOAc to afford dimethyl 2′-nitro-1,1′-biphenyl-2,4′-dicarboxylate (3.5 g).

¹H NMR (DMSO-d₆) δ: 3.59(3H, s), 3.95(3H, s), 7.37(1H, dd, J=7.6, 1.3 Hz), 7.5-7.8(3H, m), 8.03(1H, dd, J=7.7, 1.2 Hz), 8.27(1H, dd, J=8.0, 1.6 Hz), 8.57(1H, d, J=1.6 Hz).

Mass (APCI) m/e :338.3(M+Na)+.

### REFERENCE EXAMPLE 35

Dimethyl 2′-nitro-1′,1-biphenyl-2,4′-dicarboxylate (2.0 g) was dissolved in a mixture of THF (30 ml), ethanol (60 ml) and water (9 ml). To this solution were added ammonium chloride (20 mg) and iron (200 mg) and the mixture was refluxed for 5 hours. The solution was cooled to room temperature and 4N aqueous sodium hydroxide (8 ml) and water (8 ml) were added. The whole mixture was stirred for 16 hours at room temperature. Unsoluble material was removed by filtration and the filtrate was concentrated in vacuo. The filtrate was diluted with water and washed with EtOAc. The aqueous phase was acidified with conc. HCl and resulting precipitates were collected by filtration, washed with EtOAc and dried in vacuo to afford 6-oxo-5,6-dihydro-3-phenanthridine-carboxylic acid (710 mg).

¹H NMR (DMSO-d₆) δ: 7.65-7.80(2H, m), 7.86(1H, dt, J=12.2, 1.4 Hz), 8.00(1H, d, J=1.5 Hz), 8.35(1H, dd, J=7.9, 1.2 Hz), 8.45-8.60(2H, m), 11.87(1H, s).

### REFERENCE EXAMPLE 36

Under ice cooling, isobutyl chloroformate (497 mg) was added dropwise to a mixture of 6-oxo-5,6-dihydro-3-phenanthridinecarboxylic acid (725 mg) and triethylamine (613 mg) in THF (20 ml). The mixture was stirred for 1.5 hours at the same temperature. In another vessel sodium borohydride (459 mg) was dissolved in a mire of THF (10 ml) and water (20 ml) and cooled with ice. To this solution was added the above mixture over 10 minutes The mixture was stirred for 1.5 hours under ice cooling and poured into a mixture of water and EtOAc. The organic phase was separated and washed with water and brine, and then dried over magnesium sulfate. After evaporation of the solvent the residue was purified by column chromatography on silica-gel eluting with DCM-acetone to afford 3-(hydroxymethyl)-6(5H)-phenanthridinone (410 mg).

¹H NMR (DMSO-d₆) δ: 4.60(2H, d, J=5.6 Hz), 5.36(1H, t, J=5.6 Hz), 7.20(1H, dd, J=8.3, 0.9 Hz), 7.36(1H, s), 7.62(1H, t, J=7.4 Hz), 7.84(1H, t, J=8.3 Hz), 8.3-8.35(2H, m), 8.47(1H, d, J=8.1 Hz), 11.68(1H, s).

Mass (APCI) m/e:248.3(M+Na)+.

### REFERENCE EXAMPLE 37

3-(hydroymethyl)-6(5H)-phenanthridinone (370 mg) was suspended in phosphorus oxychloride (4 ml) and the mixture was stirred under reflux for 3.5 hours. The clear solution was poured into a mixture of water and chloroform and neutralized with saturated aqueous sodium hydrogen carbonate. The mixture was stirred for 30 minutes while the solution pH was maintained between 7 and 9. The organic phase was separated and washed with water and brine, and then dried over magnesium sulfate. After evaporation of the solvent the residue was purified by column chromatography on silica-gel eluting with DCM to afford 6-chloro-3-(chloromethyl)phenanthridine (256 mg).

¹H NMR (DMSO-d₆) δ: :5.03(2H, s), 7.8-8.15(4H, m), 8.45(1H, dd, J=8.2, 1.0 Hz), 8.86(1H, d, J=8.5 Hz), 8.93(1H, d, J=8.2 Hz).

Mass (APCI) m/e: 284.1, 286.1(M+Na)+.

### EXAMPLE 1

50% Pd/C catalyst (50% wet, 10 mg) was added to a solution of 2-{3-[4-phenyl-3,6-dihydro-1(2H)-pyridyl]propyl}-6(5H)-phenanthridinone (85 mg) in a mixture of THF (5 ml) and MeOH (5 ml). The mixture was stirred under hydrogen at atmospheric pressure until hydrogen gas absorption stopped. After filtration through celite and removal of the solvent, the residue was purified by column chromatography on silica-gel eluting with a mixture of chloroform and MeOH to give 2-[3-(4-phenylpiperidin-1-yl)propyl]-6(5H)-phenanthridinone (65 mg).

IR (KBr) cm⁻¹: 1666, 1608.

¹H-NMR (DMSO-d₆) δ: 1.6-2.1(8H, m), 2.2-2.5(3H, m), 2.72(2H, t, J=7.2 Hz), 2.98(2H, d, J=11.2 Hz), 7.1-7.4(7H, m), 7.63(1H, t, J=7.3 Hz), 7.84(1H, t, J=7.3 Hz), 8.23(1H, s), 8.32(1H, d, J=8.0 Hz), 8.54(1H, d, J=8.0 Hz).

Mass: 397.4 (M+H)+.

### EXAMPLE 2

4-(4-Fluorophenyl)-1,2,3,6-tetrahydropyridine hydrochloride (152 mg) was added to a solution of 2-(3-bromopropyl)-6(5H)-phenanthridinone (150 mg) in DMF (3 ml) at room temperature. Triethylamine (0.66 ml) was added to the mixture cooled in an ice bath. The whole mixture was stirred for 1 hour in the ice bath and stirred overnight at ambient temperature. The mixture was poured into a mixture of water and EtOAc. The separated organic layer was washed with brine and dried over magnesium sulfate. After evaporation of the solvent, the residue was purified by column chromatography on silica-gel eluting with a mixture of DCM and acetone and then a mixture of chloroform and MeOH to give 2-{3-[4-(4-fluorophenyl)-3,6-dihydro-1 (2H)-pyridyl]propyl}-6(5H)-phenanthridinone (78 mg).

¹H-NMR (DMSO-d₆) δ: 1.75-2.0(2H, m), 2.3-2.9(6H, m), 3.06(2H, s), 6.12(1H, s), 7.1-7.5(6H, m), 7.63(1H, t, J=7.6 Hz), 7.84(1H, t, J=7.0 Hz), 8.23(1H, s), 8.32(1H, d, J=7.5 Hz), 8.52(1H, d, J=8.0 Hz), 11.62(1H, s).

Mass: 413.13 (M+H)+.

The compounds in the following Examples 3 to 21 were obtained in a similar manner to Example 2.

### EXAMPLE 3

2-{3-[4-Phenyl-3,6-dihydro-1(2H)-pyridyl]propyl}-6(5H)-phenanthridinone

¹H-NMR (DMSO-d₆) δ: 1.8-2.0(2H, m), 2.4-2.5(4H, m), 2.6-2.8(4H, m), 3.08(2H, d, J=2.8 Hz), 6.15(1H, s), 7.1-7.5(7H, m), 7.63(1H, t, J=7.2 Hz), 7.84(1H, t, J=7.2 Hz), 8.23(1H, s), 8.32(1H, d, J=8.0 Hz), 8.53(1H, d, J=8.0 Hz), 11.61 (1H, s).

Mass: 395.3 (M+H)+.

### EXAMPLE 4

2-{3-[4-(4-Chlorophenyl)-3,6-dihydro-1(2H)-pyridyl]propyl}-6(5H)-phenanthridinone

¹H-NMR (DMSO-d₆) δ: 1.8-2.0(2H, m), 2.45-2.8(8H, m), 3.09(2H, m), 6.20(2H, m), 7.25-7.50(6H, m), 7.62(1H, t, J=7.1 Hz), 7.84(1H, t, J=7.1 Hz), 8.23(1H, s), 8.31(1H, d, J=7.9 Hz), 8.52(1H, d, J=8.0 Hz), 11.60(1H, s).

Mass: 429.2 (M+H)+.

### EXAMPLE 5

2-{3-[4-(4-Methoxyphenyl)-3,6-dihydro-1 (2H)-pyridyl]propyl}-6(5H)-phenanthridinone

¹H-NMR (DMSO-d₆) δ: 1.75-2.0(2H, m), 2.3-2.9(8H, m), 3.06(2H, s), 3.74(3H, s), 6.03(1H, s), 6.88(2H, d, J=8.6 Hz), 7.25-7.40(4H, m), 7.63(1H, t, J=7.5 Hz), 7.84(1H, t, J=7.0 Hz), 8.23(1H, s), 8.32(1H, d, J=7.7 Hz), 8.53(1H, d, J=8.1 Hz), 11.61(1H, s).

Mass: 425.0 (M+H)+.

### EXAMPLE 6

2-{3-[4-(4-Cyanophenyl)-1-piperazinyl]propyl}-6(5H)-phenanthridinone

¹H-NMR (DMSO-d₆) δ: 1.8-2.0(2H, m), 2.2-2.4(2H, m), 2.4-2.6(8H, m), 2.73(2H, t, J=7.0 Hz), 6.95-7.05(2H, m), 7.25-7.40(2H, m), 7.5-7.65(3H, m), 7.84(1H, t, J=7.0 Hz), 8.22(1H, s), 8.25-8.35(1H, m), 8.52(1H, d, J=8.0 Hz), 11.60(1H, s).

Mass: 423.3 (M+H)+.

### EXAMPLE 7

8-Chloro-2-{3-[4-(4-fluorophenyl)-3,6-dihydro-1(2H)-pyridyl]-propyl}-6(5H)-phenanthridinone

¹H-NMR (DMSO-d₆) δ: 1.75-2.0(2H, m), 2.3-2.8(8H, m), 3.07(2H, s), 6.11(1H, s), 7.0-7.5(6H, m), 7.87(1H, dd, J=8.6, 2.0 Hz), 8.0-8.4(2H, m), 8.57(1H, d, J=8.0 Hz), 11.78(1H, s).

Mass: 447.3 (M+H)+.

### EXAMPLE 8

8-Chloro-2-{3-[4-[4-(trifluoromethyl)phenyl]-3,6-dihydro-1(2H)-pyridyl]propyl}6(5H)-phenanthridinone

¹H-NMR (DMSO-d₆) δ: 1.8-2.0(2H, m), 2.4-2.8(8H, m), 3.12(2H, d, J=2.7 Hz), 6.34(1H, s), 7.2-7.4(2H, m), 7.5-7.7(5H, m), 7.84(1H, dt, J=7.2, 1.5 Hz), 8.23(1H, s), 8.25-8.35(1H, m), 8.53(1H, d, J=8.2 Hz), 11.60(1H, s).

Mass: 463.4 (M+H)+.

### EXAMPLE 9

8-Chloro-2-[3-(9-methyl-1,3,4,9-tetrahydro-2H-pyrido[3,4-b]-indol-2-yl)propyl]-6 (5H)-phenanthridinone

¹H-NMR (DMSO-d₆) δ: 1.85-2.1(2H, m), 2.4-2.9(8H, m), 3.58(3H, s), 6.35(2H, s), 6.9-7.2(2H, m), 7.25-7.5(4H, m), 7.8-8.0(1H, m), 8.2-8.4(2H, m), 8.55(1H, d, J=8.8 Hz), 11.78(1H, s).

Mass: 456.0, 458.0 (M⁺).

### EXAMPLE 10

2-[4-(4-Phenyl-3,6-dihydro-1(2H)-pyridyl)butyl]-6(5H)-phenanthridinone

¹H-NMR (DMSO-d₆) δ: 1.45-1.8(4H, m), 2.35-2.8(8H, m), 3.03(2H, d, J=2.0 Hz), 6.12(1H, s), 7.2-7.45(7H, m), 7.55-7.7(1H, m), 7.8-7.9(1H, m), 8.21(1H, s), 8.25-8.35(1H, m), 8.52(1H, d, J=8.2 Hz), 11.60(1H, s).

Mass: 409.4 (M+H)+.

### EXAMPLE 11

2-[2-(4-Phenyl-3,6-dihydro-1(2H)-pyridyl)ethoxy]-7,8,9,10-tetrahydro-6(5H)-phenanthridinone

¹H-NMR (DMSO-d₆) δ: 1.6-1.9(4H, m), 2.4-2.55(2H, m), 2.65-2.95(8H, m), 3.15-3.30(2H, m), 4.18(2H, t, J=5.8 Hz), 6.16(1H, s), 7.0-7.7(8H, m), 11.47(1H, s).

Mass: 401.3 (M+H)+.

### EXAMPLE 12

2-{2-[4-(4-Chlorophenyl)-3,6-dihydro-1(2H)-pyridyl]ethoxy}-7,8,9,10-tetrahydro-6(5H)-phenanthridinone

¹H-NMR (DMSO-d₆) δ: 1.6-1.9(4H, m), 2.4-2.55(2H, m), 2.6-3.0(8H, m), 3.2-3.4(2H, m), 4.15-4.30(2H, m), 6.15(1H, s), 7.1-7.7(7H, m), 11.49(1H, s).

Mass 435.3 (M+H)⁺.

### EXAMPLE 13

3-{2-[4-(4-Chlorophenyl)-3,6-dihydro-1(2H)-pyridyl]propyl}-7,8,9,10-tetrahydro-6(5H)-phenanthridinone

¹H-NMR (DMSO-d₆) δ: 1.65-1.90(6H, m), 2.3-2.9(10H, m), 3.05(2H, s), 3.3(2H, s), 6.19(1H, s), 7.05(1H, d, J=8.9 Hz), 7.10(1H, s), 7.36(2H, d, J=8.7 Hz), 7.46(2H, d, J=8.7 Hz), 7.70(2H, d, J=8.9 Hz), 11.50(1H, s).

Mass: 433.4 (M+H)+.

### EXAMPLE 14

3-{2-[4-(4-Chlorophenyl)-1-piperazinyl]propyl}-7,8,9,10-tetrahydro-6(5H)-phenanthridinone

¹H-NMR (DMSO-d₆) δ: 1.6-1.9(6H, m), 2.25-2.9(12H, m), 3.05-3.2(4H, m), 6.83(2H, d, J=8.9 Hz), 6.93(1H, d, J=8.2 Hz), 7.02(1H, s), 7.21(2H, d, J=8.9 Hz), 7.58(1H, d, J=8.2 Hz), 11.50(1H, s).

Mass: 435.99 (M+H)+.

### EXAMPLE 15

3-{[4-(4-Chlorophenyl)-3,6-dihydro-1(2H)-pyridyl]methyl}-7,8,9,10-tetrahydro-6(5H)-phenanthridinone

¹H-NMR (DMSO-d₆) δ: 1.6-1.9(4H, m), 2.3-2.5(2H, m), 2.65-2.9(4H, m), 3.06(2H, s), 3.4-3.5(2H, m), 3.63(2H, s), 6.19(1H, s), 7.1-7.7(7H, m), 11.53(1H, s).

Mass: 405.3(M+H)+.

### EXAMPLE 16

3-{[4-(4-Chlorophenyl)-1-piperazinyl]methyl}-7,8,9,10-tetrahydro-6(5H)-phenanthridinone

¹H-NMR (DMSO-d₆) δ: 1.6-1.9(4H, m), 2.3-2.5(2H, m), 2.65-2.9(4H, m), 3.06(2H, s), 3.4-3.5(2H, m), 3.63(2H, s), 6.19(1H, s), 7.1-7.7(7H, m), 11.53(1H, s).

Mass: 405.3(M+H)+.

### EXAMPLE 17

3-(2,3-dihydro-1H-imidazo[1,2-b]pyrazol-1-ylmethyl)-6(5H)-phenanthridinone

¹H NMR (DMSO-d₆) δ: 1.6-1.9(4H, m), 2.4-2.6(2H, m), 2.7-2.9(2H, m), 4.02(2H, d, J=8.5 Hz), 4.22(2H, d, J=8.5 Hz), 4.41(2H, s), 5.75(1H, d, J=2.6 Hz), 7.17(1H, d, J=8.0 Hz), 7.30(1H, s), 7.68(1H, d, J=8.0 Hz), 7.96(1H, d, J=2.6 Hz).

Mass (APCI) m/e: 321.2 (M+H)+.

### EXAMPLE 18

2-[(6-oxo-5,6,7,8,9,10-hexahydro-3-phenanthridinyl)-methyl]-1H-isoindol-1,3(2H)-dione

¹H NMR (DMSO-d₆) δ: 1.6-1.9(4H, m), 2.5-2.6(2H, m), 2.7-2.9(2H, m), 4.82(2H, s), 7.12(1H, dd, J=8.3, 1.5 Hz), 7.21(1H, d, J=1.5 Hz), 7.63(1H, dd, J=8.3 Hz), 7.8-8.0(4H, m), 11.47(1H, s).

Mass (APCI) m/e: 381.1(M+Na)+.

### EXAMPLE 19

3-[(9-methyl-1,3,4,9-tetrahydro-2H-beta-carbolin-2-yl)-methyl]-7,8,9,10-tetrahydro-6(5H)-phenanthridinone

¹H NMR (DMSO-d₆) δ: 1.6-1.9(4H, m), 2.4-2.6(2H, m), 2.7-3.0(4H, m), 3.5-3.7(4H, m), 3.54(3H, s), 3.82(2H, s), 6.9-7.4(6H, m), 7.65(1H, d, J=8.2 Hz), 11.57(1H, s).

Mass (APCI) m/e: 398.3(M+H)+.

### EXAMPLE 20

3-{[4-(5-methyl-2-pyridyl)-1-piperidyl]methyl}-7,8,9,10-tetrahydro-6(5H)-phenanthridinone

¹H NMR (DMSO-d₆) δ: 1.6-1.9(8H, m), 2.0-2.2(4H, m), 2.24(3H, s), 2.4-3.0(7H, m), 3.55(2H, s), 7.14(1H, d, J=7.9 Hz), 7.15(1H, d, J=7.9 Hz), 7.26(1H, d, J=2.1 Hz), 7.50(1H, dd, J=8.2,2.1 Hz), 7.62(1H, d, J=8.2 Hz), 8.31(1H, s), 11.54(1H, s).

Mass (APCI) m/e: 388.3(M+H)+.

### EXAMPLE 21

3-{[4-[4-(trifluoromethoxy)phenyl]-3,6-dihydro-1(2H)-pyridyl]methyl}-7,8,9,10-tetrahydro-6(5H)-phenanthridinone

¹H NMR (DMSO-d₆) δ: 1.6-1.9(6H, m), 2.3-2.5(2H, m), 2.55-2.75(2H, m), 2.75-2.9(2H, m), 3.0-3.15(2H, m), 3.63(2H, s), 6.20(1H, s), 7.15(1H, d, J=8.2 Hz), 7.29(1H, s), 7.31(2H, d, J=8.8 Hz), 7.54(2H, d, J=8.8 Hz), 7.63(1H, d, J=8.2 Hz), 11.55(1H, s).

Mass (APCI) m/e: 455.1(M+H)+.

### EXAMPLE 22

4-(4-Chlorophenyl)-1,2,3,6-tetrahydropyridine hydrochloride (225 mg) and triethylamine (0.91 ml) were added successively to a solution of 3-(2-bromoethyl)-7,8,9,10-tetrahydro-6(5H)-phenanthridinone (200 mg) in DMF (4 ml) at room temperature. The whole mixture was stirred overnight at ambient temperature. The mixture was poured into a mixture of water and EtOAc. The separated organic layer was washed with brine and dried over magnesium sulfate. After evaporation of the solvent, the residue was purified by column chromatography on silica-gel eluting with a mixture of DCM and acetone and then a mixture of chloroform and MeOH. A suspension of the product in MeOH (2 ml) was added with 4N hydrogen chloride (0.5 ml) to dissolve. The crystalline of the product was emerged after 1 hour. The crystalline product was collected by filtration, washed with MeOH and dried under reduced pressure to give 3-{2-[4-(4-chlorophenyl)-3,6-dihydro-1(2H)-pyridyl]ethyl}-7,8,9,10-tetrahydro-6(5H)-phenanthridinone hydrochloride (133 mg). ¹H-NMR (DMSO-d₆) δ: 1.6-1.9(4H, m), 2.45-2.55(2H, m), 2.7-2.95 (2H, m), 6.27(1H, s), 7.13(1H, d, J=8.1 Hz), 7.16(1H, s), 7.45(2H, d, J=8.7 Hz), 7.55(2H, d, J=8.7 Hz), 7.67(1H, d, J=8.1 Hz), 10.69(1H, br s), 11.65(1H, s), 3.1-4.2(10H, m).

Mass: 419.2(M+Na)⁺.

The compounds in the following Examples 23 to 39 were obtained in a similar manner to Example 22.

### EXAMPLE 23

3-{2-[4-(4-Chlorophenyl)-1-piperazinyl]ethyl}-7,8,9,10-tetrahydro-6(5H)-phenanthridinone dihydrochloride

¹H-NMR (DMSO-d₆) δ: 1.65-1.85(4H, m), 2.45-2.55(2H, m), 2.75-2.85(2H, m), 3.15-3.25(8H, m), 3.6-3.7(2H, m), 3.8-3.9(2H, m), 7.03(2H, d, J=9.0 Hz), 7.10(1H, d, J=8.4 Hz), 7.15(1H, s), 7.29(1H, d, J=8.4 Hz), 7.66(1H, d, J=8.4 Hz), 11.17(1H, br s), 11.65(1H, s).

Mass: 422.2 (M+H)+.

### EXAMPLE 24

3-[3-(4-Morpholinyl)propyl]-7,8,9,10-tetrahydro-6(5H)-phenanthridinone hydrochloride

IR (KBr) cm⁻¹: 3276, 1625, 1567.

¹H-NMR (DMSO-d₆) δ: 1.65-1.85(4H, m), 2.0-2.15(2H, m), 2.4-2.5(2H, m), 2.65-2.85(4H, m), 2.95-3.15(4H, m), 3.35-3.45(2H, m), 3.8-4.0(4H, m), 7.06(1H, dd, J=8.3, 1.6 Hz), 7.12(1H, d, J=1.6 Hz), 7.61(1H, d, J=8.3 Hz).

Mass: 327.3(M+H)+.

### EXAMPLE 25

3- [(4-Morpholinyl)methyl]-7,8,9,10-tetrahydro-6(5H)-phenanthridinone hydrochloride

IR (KBr) cm⁻¹: 3436, 1643, 1560.

¹H-NMR (DMSO-d₆) δ: 1.6-1.9(4H, m), 2.46(2H, s), 2.82(2H, s), 3.1-3.4(4H, m), 3.8-4.0(4H, m), 4.38(2H, s), 7.40(1H, s), 7.55(1H, d, J=8.0 Hz), 7.74(1H, d, J=8.0 Hz), 11.54(1H, s), 11.85(1H, s).

Mass: 299.3(M+H)+.

### EXAMPLE 26

3-{[4-Phenyl-3,6-dihydro-1(2H)-pyridyl)methyl]-7,8,9,10-tetrahydro-6(5H)-phenanthridinone hydrochloride

¹H-NMR (DMSO-d₆) δ: 1.65-1.90(4H, m), 2.45-2.55(2H, m), 2.7-2.9(4H, m), 3.5-3.9(4H, m), 4.48(2H, m), 6.16(1H, s), 7.25-7.55(7H, m), 7.78(1H, d, J=8.2 Hz), 10.78(1H, br s), 11.86(1H, s).

Mass: 371.4 (M+H)+.

### EXAMPLE 27

3-[(4-Phenylpiperidin-1-yl)methyl]-7,8,9,10-tetrahydro-6(5H)-phenanthridinone hydrochloride

¹H-NMR (DMSO-d₆) δ: 1.6-2.2(10H, m), 2.7-2.9(4H, m), 3.0-3.2(2H, m), 3.3-3.4(1H, m), 4.37(2H, d, J=4.8 Hz), 7.15-7.45(6H, m), 7.52(1H, d, J=8.2 Hz), 7.77(1H, d, J=8.2 Hz), 10.76(1H, br s), 11.84(1H, s).

Mass: 373.4 (M+H)+.

### EXAMPLE 28

3-{[4-(4-fluorophenyl)-1-piperidyl]methyl}-7,8,9,10-tetrahydro-6(5H)-phenanthridinone hydrochloride

¹H NMR (DMSO-d₆) δd: 1.6-2.2(8H, m), 2.3-2.55(2H, m), 2.7-3.2(5H, m), 3.3-3.5(2H, m), 4.37(2H, d, J=4.8 Hz), 7.1-7.3(4H, m), 7.40(1H, s), 7.56(1H, d, J=8.3 Hz), 7.76(1H, d, J=8.3 Hz), 11.04(1H, br s), 11.85(1H, s).

Mass (APCI) m/e: 391.4(M+H)+.

### EXAMPLE 29

3-{[4-(4-methoxyphenyl)-1-piperidyl]methyl}-7,8,9,10-tetrahydro-6(5H)-phenanthridinone hydrochloride

¹H NMR (DMSO-d₆) δ: 1.6-2.2(8H, m), 2.6-3.2(5H, m), 3.2-3.5(2H, m), 3.5-3.8(2H, m), 4.36(2H, d, J=4.5 Hz), 6.88(2H, d, J=8.6 Hz), 7.13(2H, d, J=8.6 Hz), 7.33(1H, s), 7.55(1H, d, J=8.3 Hz), 7.76(1H, d, J=8.3 Hz), 10.94(1H, br s), 11.85(1H, s).

Mass (APCI) m/e: 403.4 (M+H)+.

### EXAMPLE 30

3-{[4-(4-methylphenyl)-1-piperidyl]methyl}-7,8,9,10-tetrahydro-6(5H)-phenanthridinone hydrochloride

¹H NMR (DMSO-d₆) δ: 1.6-2.2(8H, m), 2.4-2.6(2H, m), 2.25(3H, s), 2.6-3.3(5H, m), 3.4-3.6(2H, m), 4.36(2H, d, J=4.7 Hz), 7.06(4H, s), 7.40(1H, s), 7.57(2H, d, J=8.3 Hz), 7.75(2H, d, J=8.3 Hz), 11.07(1H, br s), 11.85(1H, s).

Mass (APCI) m/e: 387.4(M+H)⁺.

### EXAMPLE 31

3-{14-(4-chlorophenyl)-1-piperidyl]methyl}-7,8,9,10-tetrahydro-6(5H)-phenanthridinone hydrochloride

¹H NMR (DMSO-d₆) δ: 1.6-2.2(8H, m), 2.3-2.5(2H, m), 2.7-3.2(5H, m), 3.3-3.5(2H, m), 4.37(2H, s), 7.2-7.6(6H, m), 7.75(1H, d, J=8.2 Hz), 10.95(1H, br s), 11.85(1H, s).

Mass (APCI) m/e: 407.3(M+H)+.

### EXAMPLE 32

3-({4-[4-(trifluoromethyl)phenyl]-1-piperidyl}methyl)-7,8,9,10-tetrahydro-6(5H)-phenanthridinone hydrochloride

¹H NMR (DMSO-d₆) δ: 1.6-2.4(8H, m), 2.3-2.5(2H, m), 2.7-3.3(3H, m), 3.4-3.75(4H, m), 4.39(2H, d, J=4.6 Hz), 7.4-7.5(3H, m), 7.56(1H, d, J=8.3 Hz), 7.6-7.8(3H, m), 11.05(1H, br s), 11.86(1H, s).

Mass (APCI) m/e: 441.3 (M+H)+.

### EXAMPLE 33

3-{[4-(2-pyridyl)-1-piperidyl]methyl}-7,8,9,10-tetrahydro-6(5H)-phenanthridinone dihydrochloride

¹H NMR (DMSO-d₆) δ: 1.6-1.9(4H, m), 2.1-2.6(6H, m), 2.8-3.6(7H, m), 4.40(2H, d, J=4.1 Hz), 7.34(1H, s), 7.4-8.0(4H, m), 8.51(1H, t, J=7.8 Hz), 8.80(1H, d, J=5.7 Hz), 11.39(1H, br s), 11.86(1H, s).

Mass (APCI) m/e: 374.4 (M+H)+.

### EXAMPLE 34

3-[(4-benzyl-1-piperidyl)methyl]-7,8,9,10-tetrahydro-6(5H)-phenanthridinone hydrochloride

¹H NMR (DMSO-d₆) δ: 1.5-1.9(8H, m), 2.4-2.6(2H, m), 2.7-3.0(5H, m), 3.1-3.4(2H, m), 4.27(2H, d, J=4.6 Hz), 4.64(2H, s), 7.1-7.4(6H, m), 7.51(1H, d, J=9.2 Hz), 7.73(1H, d, J=8.4 Hz), 10.79(1H, br s), 11.83(1H, s).

Mass (APCI) m/e: 387.2 (M+H)+.

### EXAMPLE 35

3-[(4-hydroxy-4-phenyl-1-piperidyl)methyl]-7,8,9,10-tetrahydro-6(5H)-phenanthridinone hydrochloride

¹H NMR (DMSO-d₆) δ: 1.6-1.9(8H, m), 2.4-2.6(2H, m), 2.75-2.95(2H, m), 3.1-3.4(2H, m), 4.42(2H, d, J=4.4 Hz), 7.2-7.5(6H, m), 7.59(1H, d, J=8.4 Hz), 7.76(1H, d, J=8.4 Hz), 11.29(1H, br s), 11.85(1H, s).

Mass (APCI) m/e: 389.2 (M+H)+.

### EXAMPLE 36

3-(1,4′-bipiperidin-1′-ylmethyl)-7,8,9,10-tetrahydro-6(5H)-phenanthridinone dihydrochloride

¹H NMR (DMSO-d₆) δ: 1.2-1.9(10H, m), 2.0-2.7(6H, m), 2.7-3.2(9H, m), 3.2-3.6(2H, m), 4.33(2H, s), 7.34(1H, s), 7.44(1H, d, J=8.0 Hz), 7.76(1H, d, J=8.0 Hz), 10.54(1H, br s), 10.84(1H, br s), 11.85(1H, s).

Mass (APCI) m/e: 380.4 (M+H)+.

### EXAMPLE 37

3-[(4-bromo-1-piperidyl)methyl]-7,8,9,10-tetrahydro-6(5H)-phenanthridinone hydrochloride

¹H NMR (DMSO-d₆) δ: 1.6-1.9(6H, m), 2.0-2.2(2H, m), 2.3-2.6(2H, m), 2.8-3.4(5H, m), 4.30(2H, d, J=2.8 Hz), 4.44(2H, d, J=4.8 Hz), 7.36(1H, s), 7.53(1H, d, J=8.3 Hz), 7.74(1H, d, J=8.3 Hz), 11.42(1H, br s), 11.85(1H, s).

Mass (APCI) m/e: 375.1, 377.1 (M+H)+.

### EXAMPLE 38

3-{[4-(5-chloro-2-pyridyl)-1-piperazinyl]methyl}-7,8,9,10-tetrahydro-6(5H)-phenanthridinone dihydrochloride

¹H NMR (DMSO-d₆) δ: 1.6-1.9(6H, m), 2.4-2.6(2H, m), 2.7-2.9(2H, m), 3.0-3.7(6H, m), 4.40(2H, s), 6.99(1H, d, J=9.2 Hz), 7.34(1H, s), 7.5-7.8(3H, m), 8.17(1H, d, J=2.6 Hz), 11.76(1H, br s), 11.85(1H, s).

Mass (APCI) m/e: 409.3 (M+H)+.

### EXAMPLE 39

3-{[4-(2-thienyl)-1-piperidyl]methyl}-7,8,9,10-tetrahydro-6(5H)-phenanthridinone hydrochloride

¹H NMR (DMSO-d₆) δ: 1.6-2.3(4H, m), 2.8-3.5(5H, m), 4.36(2H, d, J=4.9 Hz), 6.85-7.05(6H, m), 7.35-7.80(4H, m), 10.93(1H, br s), 11.86(1H, s).

Mass (APCI) m/e: 379.3(M+H)+.

### EXAMPLE 40

Under a nitrogen atmosphere, 3-bromo-7,8,9,10-tetrahydro-6(5H)-phenanthridinone (150 mg) was dissolved in dioxane (10 ml) in 20 ml of sealed tube. To this solution were added sodium tert-butoxide (1.04 g), 2,2′-bis(diphenylphophino)-1,1′-binaphthyl (101 mg) and tris(dibenzylideneacetone)dipalladium (0) (49 mg) successively. The mixture was stirred for 36 hours at 140° C. in sealed tube and then cooled to room temperature. The crude mixture was poured into a mixture of water and chloroform. The separated organic layer was washed with brine and dried over magnesium sulfate. After evaporation of the solvent, the residue was purified by column chromatography on silica-gel eluting with a mixture of DCM and acetone and then a mixture of chloroform and MeOH to give a thin yellow powder. A suspension of the yellow powder in MeOH (2 ml) was added with 4N hydrogen chloride in EtOAc (0.5 ml) to dissolve. After removal of the solvent, the resulting precipitate was washed with diethyl ether to give 3-(diethylamino)-7,8,9,10-tetrahydro-6(5H)-phenanthridinone hydrochloride (45 mg).

IR (KBr) cm⁻¹: 3401, 1643, 1558.

¹H-NMR (DMSO-d₆) δ: 1.0-1.2(6H, m), 1.65-1.9(4H, m), 2.45-2.55(2H, m), 2.7-2.9(2H, m), 3.3-3.5(4H, m), 7.15-7.75(3H, m), 11.59(1H, s).

Mass: 293.3 (M+Na)+.

### EXAMPLE 41

3-Morpholin-4-yl-7,8,9,10-tetrahydro-6(5H)-phenanthridinone (61 mg) was obtained in a similar manner to Example 40.

IR (KBr) cm⁻¹: 3420, 1641, 1554.

¹H-NMR (DMSO-d₆) δ: 1.6-1.8(4H, m), 2.4-2.5(2H, m), 2.7-2.8(2H, m), 3.1-3.2(4H, m), 3.7-3.8(4H, m), 6.68(1H, s), 6.87(1H, d, J=9.0 Hz), 7.49(1H, d, J=9.0 Hz), 11.30(1H, s).

### EXAMPLE 42

4-Hydroxy-7,8,9,10-tetrahydro-6(5H)-phenanthridinone (202 mg) was added to a solution of potassium hydroxide (63 mg) and 2-bromopyridine in dimethyl sulfoxide (20 ml) at room temperature. The mixture was stirred at 130° C. for 6 hours, cooled to room temperature and then poured into a mixture of water and EtOAc. After the pH of the solution was adjusted to 5.5 with 1N aqueous hydrogen chloride solution, an unsolvable material was removed by filtration. The separated organic layer from the filtrate was washed with brine and dried over magnesium sulfate. Evaporation of the solvent gave 3-(pyridin-2-yloxy)-7,8,9,10-tetrahydro-6(5H)-phenanthridinone (29 mg).

¹H-NMR (DMSO-d₆) δ: 1.65-1.9(4H, m), 2.4-2.55(2H, m), 2.75-3.0(2H, m), 7.05-7.35(4H, m), 7.56(1H, dd, J=7.4, 1.7 Hz), 7.8-7.9(1H, m), 8.03(1H, dd, J=4.9, 1.3 Hz), 11.20(1H, s).

Mass: 315.2 (M+Na)+.

### EXAMPLE 43

Under a nitrogen atmosphere, thiophenol (88 mg) was added to a solution of potassium tert-butoxide (89 mg) in DMF (4 ml) at 0° C. After 10 minutes, a solution of 3-[(4-bromo-1-piperidyl)methyl]-7,8,9,10-tetrahydro-6(5H)-phenanthridinone (200 mg) in DMF (2 ml) was added to the solution at the same temperature. The mixture was stirred at 60° C. for 1.5 hours and poured into a mixture of saturated aqueous sodium hydrogen carbonate and chloroform. The organic phase was separated and washed with water, brine and then dried over magnesium sulfate. After evaporation of the solvent the residue was purified by column chromatography on silica-gel eluting with DCM and acetone. The active fragments were collected and evaporated. The crystalline product was collected by filtration, washed with MeOH and dried under reduced pressure to afford 3-{[4-(phenylthio)-1-piperidyl]methyl}-7,8,9,10-tetrahydro-6(5H)-phenanthridinone hydrochloride.

¹H NMR (DMSO-d₆) δ: 1.6-2.1(8H, m), 2.3-2.6(2H, m), 2.7-3.5(7H, m), 4.30(2H, d, J=4.2 Hz), 7.2-7.6(7H, m), 7.75(1H, d, J=8.3 Hz), 11.07(1H, br s), 11.83(1H, s).

Mass (APCI) m/e: 405.2 (M+H)⁺.

### EXAMPLE 44

3-Amino-7,8,9,10-tetrahydro-6(5H)-phenanthridinone (100 mg) was dissolved in AcOH, and triethyl orthoformate (104 mg) and sodium azide (45.5 mg) were added successively. The mixture was stirred under reflux for 3 hours. The solvent was evaporated in vacuo and the residue was diluted with a mixture of saturated aqueous sodium hydrogen carbonate and chloroform. The organic phase was separated and washed with water, brine and then dried over magnesium sulfate. Evaporation of the solvent afforded 3-(1H-tetrazol-1-yl)-7,8,9,10-tetrahydro-6(5H)-phenanthridinone (55 mg).

¹H NMR (DMSO-d₆) δ: 1.6-1.9(4H, m), 2.4-2.6(2H, m), 2.8-2.9(2H, m), 7.68(1H, dd, J=8.7, 2.2 Hz), 7.80(1H, d, J=2.2 Hz), 7.90(1H, d, J=8.7 Hz), 10.18(1H, s), 11.91(1H, s).

Mass (APCI) m/e: 290.2 (M+Na)+.

### EXAMPLE 45

4-Fluoro-2-(6-oxo-5,6,7,8,9,10-hexahydro-3-phenanthridinyl)-1H-isoindol-1,3(2H)-dione was obtained in a similar manner to Reference Example 32.

¹H NMR (DMSO-d₆) δ: 1.6-1.9(4H, m), 2.3-2.5(2H, m), 2.8-2.9(2H, m), 7.26(1H, dd, J=8.6, 1.9 Hz), 7.39(1H, d, J=1.9 Hz), 7.65-8.05(4H, m), 11.81(1H, s).

Mass (APCI) m/e: 385.0(M+Na)⁺.

### EXAMPLE 46

4-Phenylpiperazine hydrochloride (75 mg) and triethylamine (154 mg) were added successively to a solution of 6-chloro-3-(chloromethyl)phenanthridine (100 mg) in DMF (4 ml) at room temperature. The whole mixture was stirred overnight at ambient temperature. The mixture was poured into a mixture of water and chloroform and the aqueous layer was separated. The organic layer was washed with brine and dried over magnesium sulfate. After evaporation of the solvent the residue was purified by column chromatography on silica-gel eluting with DCM and acetone. After evaporation of the solvent, the residue was suspended in a mixture of 4N aqueous HCl (3 ml) and ethanol (3 ml). The resulting crystalline product was collected by filtration, washed with MeOH and dried under reduced pressure to afford 3-[(4-phenyl-1-piperidyl)methyl]-6(5H)-phenanthridinone hydrochloride (144 mg).

¹H NMR (DMSO-d₆) δ: 1.8-2.3(4H, m), 2.80(1H, m), 3.0-3.3(2H, m), 3.4-3.6(2H, m), 4.41(2H, d, J=4.7 Hz), 7.2-7.35(5H, m), 7.50(1H, s), 7.65-7.75(2H, m), 7.89(1H, t, J=8.0 Hz), 8.35(1H, t, J=7.9 Hz), 8.45-8.6(2H, m), 11.07(1H, br s), 11.94(1H, 1).

Mass (APCI) m/e: 369.3(M+H)+.

The compounds in the following Examples 47 to 62 were obtained in a similar manner to Example 46.

### EXAMPLE 47

3-[(4-phenyl-3,6-dihydro-1(2H)-pyridyl)methyl]-6(5H)-phenanthridinone hydrochloride

¹H NMR (DMSO-d₆) δ: 2.6-3.1(2H, m), 3.5-4.0(4H, m), 4.51(2H, s), 6.17(1H, s), 7.0-7.5(6H, m), 7.6-7.75(2H, m), 7.8-7.9(1H, m), 8.35(1H, d, J=7.9 Hz), 8.5-8.6(2H, m), 11.1(1H, br s), 11.94(1H, s). Mass (APCI) m/e: 367.4 (M+H)+.

### EXAMPLE 48

3-[(4-phenyl-1-piperazinyl)methyl]-6(5H)-phenanthridinone hydrochloride

¹H NMR (DMSO-d₆) δ: 3.1-3.5(6H, m), 3.7-3.9(2H, m), 4.48(2H, s), 6.86(1H, t, J=7.2 Hz), 6.99(1H, d, J=8.1 Hz), 7.2-7.3(2H, m), 7.51(1H, s), 7.65-7.75(2H, m), 7.89(1H, t, J=7.0 Hz), 8.34(1H, d, J=7.9 Hz), 8.45-8.60(2H, m), 11.60(1H, br s), 11.95(1H, s).

Mass (APCI) m/e: 370.4 (M+H)+.

### EXAMPLE 49

3-{[4-(4-fluorophenyl)-1-piperazinyl]methyl}-6 (5H)-phenanthridinone hydrochloride

¹H NMR (DMSO-d₆) δ: 3. 1-3.8(8H, m), 4.48(2H, s), 6.95-7. 15(4H, m), 7.51(1H, s), 7.65-7.75(2H, m), 7.85-7.95(1H, m), 8.34(1H, d, J=7.9 Hz), 8.45-8.60(2H, m), 11.58(1H, br s), 11.95(1H, s).

Mass (APCI) m/e: 388.3(M+H)+.

### EXAMPLE 50

3-{[4-(2-pyridyl)-1-piperidyl]methyl}-6(5H) -phenanthridinone dihydrochloride

¹H NMR (DMSO-d₆) δ: 2.2-2.4(4H, m), 3.1-3.6(4H, m), 4.44(2H, d, J=3.3 Hz), 7.12(1H, s), 7.35-7.95(5H, m), 8.33(1H, d, J=7.8 Hz), 8.45-8.60(3H, m), 8.79(1H, d, J=5.2 Hz), 11.48(1H, br s), 11.94(1H, s).

Mass (APCI) m/e: 370.3(M+H)+.

### EXAMPLE 51

3-{[4-(4-nitrophenyl)-1-piperazinyl]methyl}-6(5H)-phenanthridinone hydrochloride

¹H NMR (DMSO-d₆) δ: 3.1-3.8(6H, m), 4.1-4.3(2H, m), 4.46(2H, s), 7.10(2H, d, J=9.3 Hz), 7.47(1H, s), 7.6-7.75(2H, m), 7.89(1H, t, J=7.1 Hz), 8.12(2H, d, J=9.3 Hz), 8.34(1H, d, J=7.8 Hz), 8.45-8.60(2H, m), 11.50(1H, br s), 11.95(1H, s).

Mass (APCI) m/e: 437.2(M+Na)+.

### EXAMPLE 52

3-{[4-(5-chloro-2-pyridyl)-1-piperazinyl]methyl}-6(5H)-phenanthridinone dihydrochloride

¹H NMR (DMSO-d₆) δ: 3.0-3.5(6H, m), 4.2-4.6(2H, m), 4.57(2H, s), 6.99(1H, d, J=9.1 Hz), 7.47(1H, s), 7.65-7.75(3H, m), 7.89(1H, t, J=7.0 Hz), 8.17(1H, d, J=9.3 Hz), 8.34(1H, d, J=7.8 Hz), 8.45-8.60(2H, m), 11.69(1H, br s), 11.94(1H, s).

Mass (APCI) m/e: 405.2(M+H)+.

### EXAMPLE 53

3-{[4-(4-chlorophenyl)-1-piperidyl]methyl}-6(5H)-phenanthridinone hydrochloride

¹H NMR (DMSO-d₆) δ: 2.00(4H, m), 2.83(1H, m), 3.13(2H, m), 3.65(2H, m), 4.40(2H, s), 7.26(1H, d, J=8.4 Hz), 7.40(1H, d, J=8.4 Hz), 7.47(1H, s), 7.61-7.73(2H), 7.90(1H, t, J=7.2 Hz), 8.34(1H, d, 7.6 Hz), 8.49-8.60(2H), 10.87(1H, brs), 11.94(1H, s).

Mass (APCI) m/e: 403 (M+H)+.

### EXAMPLE 54

3-{[4-(4-methoxyphenyl)-1-piperidyl]methyl}-6(5H)-phenanthridinone hydrochloride

¹H NMR (DMSO-d₆) δ: 1.90-1.93(2H, m), 2.03-2.09(2H, m), 2.74(1H, m), 3.08-3.11(2H, m), 3.42-3.51(2H, m), 3.72(3H, s), 4.40(2H, s), 6.88(2H, d, J=8.6 Hz), 7.14(2H, d, J=8.6 Hz), 7.49(1H, s), 7.64-7.71(2H, m), 7.89(1H, t, J=7.8 Hz), 8.34(1H, d, J=7.8 Hz), 8.51(1H, d, J=8.4 Hz), 8.57(1H, d, J=8.4 Hz), 10.94(1H, brs), 11.92(1H, s).

Mass (APCI) m/e: 399(M+H)+.

### EXAMPLE 55

3-{[4-(4-fluorophenyl)-1-piperidyl]methyl}-6(5H)-phenanthridinone hydrochloride

¹H NMR (DMSO-d₆) δ: 1.98(4H), 2.83(1H, m), 3.13(2H, m), 3.48(2H, m), 4.40(2H, s), 7.11-7.31(4H, m), 7.49(1H, s), 7.64-7.73(2H), 7.86(1H, t, J=7.0 Hz), 8.35(1H, dd, J=1.0, 8.0 Hz), 8.50-8.60(2H), 1.00(1H, brs), 11.95(1H, s).

Mass (APCI) m/e: 387(M+H)+.

### EXAMPLE 56

3-[4-(4-hydroxy-4-phenyl-1-piperidyl)methyl]-6(5H)-phenanthridinone hydrochloride

¹H NMR (DMSO-d₆) δ: 1.75-3.41(6H), 4.48(2H, s), 7.22-7.50(6H, m), 7.62-7.69(2H, m), 7.90(1H, t, J=7.0 Hz), 8.34(1H, d, J=6.8 Hz), 8.50-8.60(2H, m), 10.87(1H, brs), 11.95(1H, s).

Mass (APCI) m/e: 385 (M+H)+.

### EXAMPLE 57

3-{[4-(4-chlorophenyl)-3,6-dihydro-1(2H)-pyridyl]methyl}-6(5H)-phenanthridinone hydrochloride

¹H NMR (DMSO-d₆) δ: 2.70-2.88(2H), 3.38-3.80(4H), 4.51(2H, s), 6.22(1H, s), 7.42-7.52(5H, m), 7.69(2H, t, J=7.8 Hz), 7.86-7.94(1H, m), 8.35(1H, dd, J=1.2 Hz, 7.8 Hz), 8.50-8.60(2H, m), 11.22(1H, brs), 11.95(1H, s).

### EXAMPLE 58

3-{[4-(4-methylphenyl)-3,6-dihydro-1(2H)-pyridyl]methyl}-6(5H)-phenanthridinone hydrochloride

¹H NMR (DMSO-d₆) δ: 2.29(3H, s), 2.70-2.89(2H,m), 3.37(1H), 3.60(1H, m), 3.80(2H), 4.50(2H, s), 6.13(1H, s), 7.19(1H, d, J=8.2 Hz), 7.37(1H, J=8.2 Hz), 7.53(1H, s), 7.64-7.73(2H, m), 7.86-7.94(1H, m), 8.35, (1H, dd, J=2.0, 7.4 Hz), 8.50-8.60(2H, m), 11.16(1H, brs), 11.94(1H, s).

### EXAMPLE 59

3-(1,4′-bipiperidin-1′-ylmethyl)-6(5H)-phenanthridinone dihydrochloride

¹H NMR (DMSO-d₆) δ: 1.23-1.38(2H), 1.60-1.81(5H), 2.00-2.27(4H), 2.94-3.05(4H), 3.20-3.49(4H), 4.37(2H, s), 7.44(1H, s), 7.58(1H, d, J=7.8 Hz), 7.65-7.93(2H, m), 8.34(1H, d, J=7.8 Hz), 8.47-8.60(2H), 10.72(1H, brs), 11.07(1H, brs), 11.93(1H, s).

### EXAMPLE 60

3-(1-piperidylmethyl)-6(5H)-phenanthridinone

¹H NMR (DMSO-d₆) δ: 1.40-1.41(2H, m), 1.50-1.53(4H, m), 2.36(4H, brs), 3.49(2H, s), 7.19(1H, d, J=8.2 Hz), 7.32(1H, s), 7.62(1H, t, J=8.0 Hz), 7.84(1H, t, J=8.0 Hz), 8.30-8.33(2H, m), 8.47(1H, d, J=8.2 Hz) 11.63(1H, brs)

### EXAMPLE 61

3-{[(3S,5S)-3,5-dimethyl-4-morpholinyl]methyl}-6(5H)-phenanthridinone hydrochloride

¹H NMR (DMSO-d₆) δ: 1.31-1.41(6H, m), 3.19-3.22(1H, m), 3.62-3.72(3H), 3.92-4.03(2H), 4.15-4.26(1H, m), 4.80(1H, dd, J=3.5, 13.6 Hz), 7.51(1H, s), 7.68(1H, t, J=7.5 Hz), 7.80-7.93(2H), 8.34(1H, d, J=8.8 Hz), 8.49-8.59(2H), 11.23(1H, brs), 11.87(1H, s)

### EXAMPLE 62

3-(4-morpholinylmethyl)-6(5H)-phenanthridinone hydrochloride

¹H NMR (DMSO-d₆) δ: 3.1-4.1(4H, m), 4.35(2H, s), 7.48(1H, d, J=1.2 Hz), 7.6-7.8(2H, m), 7.89(1H, td, J=7.6, 1.4 Hz), 8.34(1H, dd, J=7.9, 1.2 Hz), 8.49(1H, d, J=8.4 Hz), 8.57(1H, d, J=8.1 Hz).

Mass (APCI) m/e: 295.3 (M+H)+.

### EXAMPLE 63

3-{[4-(5-methyl-2-pyridyl)-1-piperidyl]methyl}-6(5H)-phenanthridinone was obtained in a similar manner to Example 2.

¹H NMR (DMSO-d₆) δ: 1.8-1.9(4H, m), 2.1-2.2(2H, m), 2.24(3H, s), 2.6-2.8(1H, m), 3.4-3.6(2H, m), 3.56(2H, s), 7.15-7.25(2H, m), 7.37(1H, s), 7.48-7.85(3H, m), 8.25-8.50(4H, m), 11.63(1H, s).

Mass (APCI) m/e: 384.2(M+H)+.

1. A compound of the formula (I): 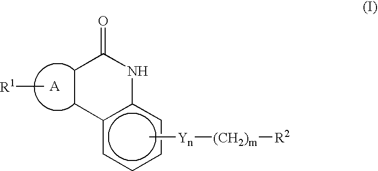
wherein
ring A is a carbocyclic group,
R¹ is hydrogen or a halogen atom or a lower alkyl group,
R² is a di(lower)alkylamino group or N-containing heterocyclic group, among which the N-containing heterocyclic group may be substituted with one or more substituent(s),
Y is an oxygen or sulfur atom,
n is an integer from 0 to 2, and
m is an integer from 0 to 4,
or its prodrug, or their salt.

2. A compound of claim 1, wherein
ring A is a cyclo(lower)alkane ring or aromatic hydrocarbon ring,
R¹ is hydrogen or a halogen atom,
R² is a di(lower)alkylamino group, a N-containing heterocyclic group, among which the N-containing heterocyclic group may be substituted with one or more substituent(s),
Y is an oxygen or sulfur atom,
n is an integer of 0 or 1, and
m is an integer from 0 to 4,
or a salt thereof.

3. A compound of claim 2, wherein R² is tetrahydropyridyl, pyridyl, piperidyl, piperazinyl, morpholinyl or pyrido[3,4-b]indolyl, tetrazolyl, isoindolidinyl, each of which may be substituted with one or more substituent(s).

4. A compound of claim 3, wherein the ring A is a cyclohexane ring and R¹ is hydrogen atom.

5. A compound of claim 4, wherein Y is an oxygen atom and m is an integer from 0 to 3.

6. A compound of claim 3, wherein the ring A is a benzene ring, n is 0 and m is an integer 1 to 4.

7. A compound of claim 6, wherein R² is morpholinyl and m is 1.

8. A pharmaceutical composition comprising a compound of the formula (I): 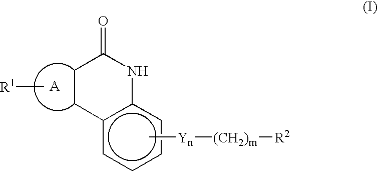
wherein the ring A, R¹, R², Y, n and m are the same meanings as defined in claim 1,
its prodrug or a pharmaceutically acceptable salt thereof in admixture with a pharmaceutically acceptable carrier.

9. The pharmaceutical composition of claim 8 which is used for treating or preventing diseases ascribed by excess activation of PARP.

10. The pharmaceutical composition of claim 9 wherein diseases ascribed by excess activation of PARP are tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; Amyotrophic Lateral Scleosis (ALS); Huntington's disease; schizopherenia; chronic pain; ischemia and neuronal loss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; atheroscleosis; osteoarthritis; osteoporosis; muscular dystrophy; degenerative diseases of skeletal muscle involving replicative senescence; age-related macular degeneration; immune senescence; AIDS; and other immune senescencediseases; inflammatory bowel disorders (e.g., colitis); arthritis; diabetes; endotoxic shock; septic shock; and/or tumor.

11. A method for treating or preventing diseases ascribed by excess activation of PARP by administering a compound of the formula (I): 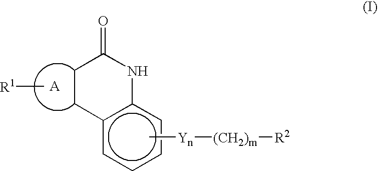
wherein the ring A, R¹, R², Y, n and m are the same meanings as defined in claim 1,
its prodrug, or a pharmaceutically acceptable salt thereof in an effective amount to inhibit PARP activity, to human being or an animal who needs to be treated or prevented.

12. A use of the compound of claim 1 as a medicament.

13. A use of the compound of claim 1 for preparing a medicament for treating or preventing diseases ascribed by excess activation of PARP.

14. The use of claim 13 wherein diseases ascribed by excess activation of PARP are tissue damage resulting from cell damage or death due to necrosis or apoptosis; neural tissue damage resulting from ischemia and reperfusion injury, neurological disorders and neurodegenerative diseases; neurodegenerative diseases; head trauma; stroke; Alzheimer's disease; Perkinson's disease; epilepsy; Amyotrophic Lateral Scleosis (ALS); Huntington's disease; schizopherenia; chronic pain; ischemia and neuronal loss following hypoxia; hypoglycemia; ischemia; trauma; nervous insult; previously ischemic heart or skeleton muscle tissue; radiosensitizing hypoxic tumor cells; tumor cells from recovering from potentially lethal damage of DNA after radiation therapy; skin aging; atheroscleosis; osteoarthritis; osteoporosis; muscular dystrophy; degenerative diseases of skeletal muscle involving replicative senescence; age-related macular degeneration; immune senescence; AIDS; and other immune senescencediseases; inflammatory bowel disorders (e.g., colitis); arthritis; diabetes; endotoxic shock; septic shock; and tumor.
